SEQUENCE LISTING (30 sequences)

SEQ ID NO: 1 - DNA, 75 bp, synthetic construct
  source 1..75: mol_type = other DNA
atgatacggcgaccaccgagatctacactctttccctacacgacgctcttccgatctgatgtccacgaggtctct

SEQ ID NO: 2 - DNA, 77 bp, synthetic construct
  source 1..77: mol_type = other DNA
aatgatacggcgaccaccgagatctacactctttccctacacgacgctcttccgatctcgatgtccacgaggtctct

SEQ ID NO: 3 - DNA, 78 bp, synthetic construct
  source 1..78: mol_type = other DNA
aatgatacggcgaccaccgagatctacactctttccctacacgacgctcttccgatctgcgatgtccacgaggtctct

SEQ ID NO: 4 - DNA, 79 bp, synthetic construct
  source 1..79: mol_type = other DNA
aatgatacggcgaccaccgagatctacactctttccctacacgacgctcttccgatctagcgatgtccacgaggtctct

SEQ ID NO: 5 - DNA, 80 bp, synthetic construct
  source 1..80: mol_type = other DNA
aatgatacggcgaccaccgagatctacactctttccctacacgacgctcttccgatctcaacgatgtccacgaggtctct

SEQ ID NO: 6 - DNA, 82 bp, synthetic construct
  source 1..82: mol_type = other DNA
aatgatacggcgaccaccgagatctacactctttccctacacgacgctcttccgatcttgcaccgatgtccacgaggtctct

SEQ ID NO: 7 - DNA, 83 bp, synthetic construct
  source 1..83: mol_type = other DNA
aatgatacggcgaccaccgagatctacactctttccctacacgacgctcttccgatctacgcaacgatgtccacgaggtctct

SEQ ID NO: 8 - DNA, 84 bp, synthetic construct
  source 1..84: mol_type = other DNA
aatgatacggcgaccaccgagatctacactctttccctacacgacgctcttccgatctgaagacccgatgtccacgaggtctct

SEQ ID NO: 9 - DNA, 88 bp, synthetic construct
  source 1..88: mol_type = other DNA
aatgatacggcgaccaccgagatctacactctttccctacacgacgctcttccgatctgaagacccttgtggaaaggacgaaacaccg

SEQ ID NO: 10 - DNA, 91 bp, synthetic construct
  source 1..91: mol_type = other DNA
caagcagaagacggcatacgagatnnnnnnnngtgactggagttcagacgtgtgctcttccgatctccaattcccactcctttcaagacct

SEQ ID NO: 11 - DNA, 23 bp, synthetic construct
  source 1..23: mol_type = other DNA
aaaggcgcgccgagggcctattt

SEQ ID NO: 12 - DNA, 33 bp, synthetic construct
  source 1..33: mol_type = other DNA
ttttttggtctcccggtgtttcgtcctttccac

SEQ ID NO: 13 - DNA, 25 bp, synthetic construct
  source 1..25: mol_type = other DNA
gttccctgcaggaaaaaagcaccga

SEQ ID NO: 14 - DNA, 62 bp, synthetic construct
  source 1..62: mol_type = other DNA
aaaaaaggtctctaccgnnnnnnnnnnnnnnnnnnnngttttagagctagaaatagcaagtt

SEQ ID NO: 15 - DNA, 20 bp, synthetic construct
  source 1..20: mol_type = other DNA
gcactaccagagctaactca

SEQ ID NO: 16 - DNA, 20 bp, synthetic construct
  source 1..20: mol_type = other DNA
aatgaccccagctcagggaa

SEQ ID NO: 17 - DNA, 20 bp, synthetic construct
  source 1..20: mol_type = other DNA
ccagtttctggataaaggat

SEQ ID NO: 18 - DNA, 20 bp, synthetic construct
  source 1..20: mol_type = other DNA
agaccttctcttcacactgg

SEQ ID NO: 19 - DNA, 20 bp, synthetic construct
  source 1..20: mol_type = other DNA
gctgggcctgggctacacgt

SEQ ID NO: 20 - DNA, 20 bp, synthetic construct
  source 1..20: mol_type = other DNA
gatgtacaacaactgtgaag

SEQ ID NO: 21 - DNA, 20 bp, synthetic construct
  source 1..20: mol_type = other DNA
ggccaataaggagtgccagg

SEQ ID NO: 22 - DNA, 20 bp, synthetic construct
  source 1..20: mol_type = other DNA
gcctgggaaagagctccccg

SEQ ID NO: 23 - DNA, 20 bp, synthetic construct
  source 1..20: mol_type = other DNA
tgaggatgacatacatgacg

SEQ ID NO: 24 - DNA, 43 bp, synthetic construct
  source 1..43: mol_type = other DNA
gtacaagtaannnnnnnngatgtccacgaggtctctgctagcg

SEQ ID NO: 25 - DNA, 43 bp, synthetic construct
  source 1..43: mol_type = other DNA
aattcgctagcagagacctcgtggacatcnnnnnnnnttactt

SEQ ID NO: 26-30 - skipped (5 entries)

(19) United States
(12) Patent Application Publication
(10) Pub. No.: US 2026/0132454 A1
(43) Pub. Date: May 14, 2026

(54) MICROFLUIDIC PLATFORM FOR THE STUDY OF MOLECULAR AND/OR CELLULAR INTERACTIONS

(71) Applicant: The Brigham and Women's Hospital, Inc., Boston, MA (US)

(72) Inventors: Francisco J. Quintana, Needham, MA (US); Michael A. Wheeler, Roslindale, MA (US); Iain C. Clark, Berkeley, CA (US)

(21) Appl. No.: 18/861,947

(22) PCT Filed: May 2, 2023
(86) PCT No.: PCT/US2023/020736

§ 371 Date: Oct. 31, 2024

Related U.S. Application Data
(60) Provisional application No. 63/337,369, filed on May 2, 2022.

(51) Int. Cl. C12Q 1/6869; C12N 9/22; C12N 15/10; C12N 15/11; C12Q 1/34; C12Q 1/686

(52) U.S. Cl. CPC C12Q 1/6869; C12N 9/226; C12N 15/1055; C12N 15/1058; C12N 15/111; C12Q 1/34; C12Q 1/686; C12N 2310/20; G01N 2333/922

(57) ABSTRACT

Described herein are high-throughput platforms and methods of using the platforms that enable the identification of cellular and molecular interactions in high-throughput screens.

### CLAIM OF PRIORITY

This application claims the benefit of U.S. Patent Application Ser. No. 63/337,369 filed on May 2, 2022. The contents of which are hereby incorporated by reference.

### SEQUENCE LISTING

This application contains a Sequence Listing that has been submitted electronically as an XML file named “29618-0380US1_SL_ST26.XML.” The XML file, created on Jun. 20, 2025, is 24,780 bytes in size. The material in the XML file is hereby incorporated by reference in its entirety.

### TECHNICAL FIELD

Described herein are high-throughput platforms and methods of using the platforms that enable the identification of cellular and molecular interactions in high-throughput screens.

### BACKGROUND

Screening platforms, such as forward genetic screens based on the CRISPR/Cas9 system, are tools used to identify genes that control biologic processes of interest (17-24). However, limitations linked to the high throughput co-culture and screening of perturbed single cells hamper their use to study cell-cell interactions. Accordingly, there is a need for high-throughput systems and methods for identifying and understanding interactions not only between cells but also between various molecules.

### SUMMARY

This disclosure describes the Systematic Perturbation of Encapsulated Associated Cells followed by Sequencing (SPEAC-seq) protocol, which is a high throughput platform that enables screening of various interaction mechanisms.

Accordingly, provided herein are methods for studying interactions between particles, the method comprising: obtaining a plurality of first particles, wherein each member of the plurality of first particles comprises one or more members of a library of interest; obtaining a plurality of second particles, each second particle comprising a reporter molecule; producing a plurality of droplets by encapsulating both a single first particle and a single second particle within an individual droplet; incubating the plurality of droplets to allow for at least one interaction to occur between the encapsulated first and second particles; selecting droplets that contain a reporter signal corresponding to the at least one interaction; and analyzing the selected droplets to study the at least one interaction.

In some embodiments, the library of interest is a genetic perturbation library, a phage display library, a library of chemically diverse small particles, a library of mutant versions of a protein, or a library of gene promoters or enhancers.

In some embodiments, the genetic perturbation library is a CRISPR/Cas9 library.

In some embodiments, the first and second particles are cells and the second cell is a partner cell to the first cell.

In some embodiments, the reporter molecule is located in the second particle.

In some embodiments, the reporter molecule enables optical detection.

In some embodiments, the reporter molecule is a fluorescent protein.

In some embodiments, the fluorescent protein is Green Fluorescent Protein (GFP).

In some embodiments, the methods further comprise, prior to encapsulation, staining the first and second particles with a viability dye.

In some embodiments, the methods further comprise staining the droplets with a reference dye.

In some embodiments, the droplets are sorted using multi-color optics.

In some embodiments, the droplets are sorted using 3-color optics.

In some embodiments, the 3-color optics is used to select droplets based on size of a droplet, presence of an activated reporter within a droplet, and a presence of both of the two particle types within a single droplet.

In some embodiments, the droplets are further sorted using dielectrophoresis, electromagnetism, fluid flow forces, or physical partitions.

In some embodiments, the droplets are sorted using electromagnetism.

In some embodiments, the droplets are sorted using dielectrophoresis.

In some embodiments, analyzing the droplets comprises: breaking the droplets; isolating perturbations; assessing a readout of the perturbations; and filtering the perturbations.

In some embodiments, the droplets are broken through chemical means or physical means.

In some embodiments, the droplets are broken using a combination of at least one freeze-thaw cycle and perfluorooctanol (PFO).

In some embodiments, the droplets are broken by applying an electric field to break the droplets.

In some embodiments, assessing the readout comprises conducting PCR amplification and sequencing of the perturbations.

In some embodiments, filtering the perturbations comprises comparing the results from the readout against known datasets and identifying unique perturbations that have not yet been previously identified.

The use of numerical values in the various ranges specified in this application, unless expressly indicated otherwise, are stated as approximations as though the minimum and maximum values within the stated ranges are both preceded by the word “about.” In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein “a” and “an” refer to one or more.

As used herein, the terms “comprising,” “comprise” or “comprised,” and variations thereof, are open ended and do not exclude the presence of other elements not identified. In contrast, the term “consisting of” and variations thereof is intended to be closed, and excludes additional elements in anything but trace amounts.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

As used herein, “plurality” means more than one (e.g., 5, 10, 15, 20, 25, 30, 40, 45, 50, 100, 200, 300, 400, 500, 1000, 10000, 100000, 1000000, 10000000, 100000000, 1000000000).

As used herein, “molecules” refer to substances that have similar physical and chemical properties to one another (e.g., nucleic acids, proteins, small molecules, or macromolecules).

As used herein, a “reporter molecule” or a “reporter” is a molecule that can serve as a proxy for a condition/interaction of interest. For example, it is a molecule that is either activated or deactivated under specific conditions. Reporter molecules can be used either to help track the physical location of another molecule or to monitor expression of a gene. Reporter molecules include agents that can be detected optically, such as fluorescent molecules, bioluminescent molecules, and the like.

As used herein, a “reporter signal” indicates the activation or deactivation of a reporter molecule. For example, a few common reporter molecules are listed here and the way in which their activation/deactivation is measured are noted: (a) β-galactosidase (GAL), which hydrolyzes colorless galactosides to yield colored products; (b) β-glucuronidase, which hydrolyzes colorless glucuronides to yield colored products; (c) β-lactamase, which catalyzes hydrolysis of a cephalosporin monitored by a change in fluorescence emission of a substrate; (d) firefly luciferase, which oxidizes luciferin, emitting photons, and Renilla luciferase, which catalyzes oxidation of coelentrazine, leading to bioluminescence; and (e) fluorescent reporter molecules, such as, green fluorescent protein (GFP), yellow fluorescent protein (YFP), or red fluorescent protein (RFP), which fluoresce, due to energy transfer, on irradiation.

As used herein, “interaction” refers to two or more particles that cooperate with one another. This can include physical and functional interactions. For example, a physical interaction is one where two or more particles physically cooperate (e.g., bind) with one another. A functional interaction refers to situations where two or more particles cooperate with one another, which then results in an alteration of activity of one or more of the particles, thereby resulting in other downstream effects. In some embodiments, a functional interaction can be assessed using a suitable activity assays (e.g., one that accesses biological readouts, affinity assays, protein binding assays, mass spectrometry, etc.). For example, in some embodiments in the present invention, once droplets are sorted and their contents isolated, biological readouts such as the foregoing can be performed to understand the interactions between the studied particles.

As used herein, the phrase “partner particles” refer to particles that, under normal biological conditions, interact with one another. For example, in one instance the two partner particles are cells. In this situation, for example in one embodiment, one cell releases a protein that activates another cell. These two cells are considered partner particles or partner cells.

As used herein, the phrases “small molecule” or “small molecule compound” are used interchangeably and are used to refer to low molecular weight organic compounds that have been chemically synthesized.

As used herein, a “perturbation” refers to variants (e.g., variants of a gene or variants of protein). Thus, “systematic perturbation” refers to variants that are generated based according to a fixed system (in one example, this approach includes alanine screening).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic flowchart showing the overall workflow of the platform described herein.

FIG. 2A is a schematic diagram showing how cells are co-encapsulated using microfluidics inside picoliter water-in-oil droplets (left) and how the droplets are co-incubated.

FIG. 2B is a scatterplot quantifying dye leakage between droplets showing approximately 1:2,000 droplets contained both dyes in phenol red-free media after 24 hours of culture at 37° C.

FIG. 2C is a schematic diagram of a cell encapsulation co-flow device showing the location of cell and oil inlets. This device was used to encapsulate two distinct cell populations, those expressing: (i) a reporter and (ii) a perturbation derived from a CRISPR/Cas9 lentivirus library. Soluble factors (TNFα, IL-1β) were added using a central channel when desired.

FIG. 2D are photographic images of a co-flow device encapsulating cells (left), and the resulting collected droplets (right).

FIG. 2E is a schematic diagram showing how co-incubated cell pairs are monitored based on their fluorescence using a 3-color droplet cytometric system and are then sorted with dielectrophoresis to isolate cell-cell pairs.

FIG. 2F is a schematic diagram of a concentric droplet sorter showing the location of droplets, bias oil, sorting electrode, and the waste and sorted outlet channels.

FIG. 2G is a photograph of the microdroplet generating and sorting system including: a custom droplet cytometer and software for detecting, gating, and sorting drops containing cell pairs based on fluorescence.

FIG. 3A is a bar graph quantifying primary astrocyte survival in droplets over a period of 72 hours. Calcein-stained cells were encapsulated in droplets, cultured for the indicated period of time, droplets were broken, and cells were analyzed by FACS to measure the fraction of live cells.

FIG. 3B are graphs showing droplet cytometry of EGFP⁺ droplets following stimulation of p65^{EGFP} reporter astrocytes with 10⁻¹ μg/mL (left) or 10⁻⁷ μg/mL (middle) TNFα and IL-1β at 24 hours post-encapsulation. Right: frequency of EGFP⁺ droplets as a function of TNFα and IL-1β concentration.

FIG. 3C are bar graphs showing an analysis of pro-inflammatory cytokine expression upon a 24-h pre-stimulation with a subthreshold dose of IL-1β/TNFα (0.1 pg/mL) with or without subsequent IL-6 or GM-CSF stimulation. n=5-6 per condition. Unpaired two-tailed t-test.

FIG. 3D are bar graphs showing the quantification of IL-1β and TNFα by ELISA in media conditioned by LPS-activated microglia. n=3 per group. Unpaired two-tailed t-test.

FIG. 3E is a schematic diagram showing an encapsulated microglial cell and a bar graph showing frequency of EGFP⁺ droplets following co-incubation of sub-threshold stimulated or unstimulated p65^{EGFP} astrocytes with microglia conditioned media or control media at 4-, 9- and 24-hours post-encapsulation.

FIG. 4A (left) is a micrograph showing cells co-incubated within droplets remain isolated from neighboring cell pairs and interact via direct contact and/or secreted soluble factors. Cell loading (as shown in a middle graph and schematic on right) determines the probability that a drop contains each cell type. Cell loading was set to favor a single cell containing a CRISPR/Cas9 perturbation.

FIG. 4B is a graph showing droplet cytometric time trace data showing presence of droplet (PMT3, low sustained intensity), cell 2 (PMT 3, sharp intensity peak), EGFP reporter (PMT1), and cell 1 (PMT2). An inert CY5 tracer dye was added to detect, and gate drops of the correct size (left). The schematic (right) shows possible combinations of cell-cell pairings and their corresponding droplet fluorescence traces.

FIG. 4C is a schematic of the gating strategy results showing how cell-cell pairs were identified by sequentially gating droplets that 1) were the correct size, 2) contained an activated reporter cell (astrocyte), and 3) were paired with the desired cell-cell pair (astrocyte-microglia) and sorted such that only droplets containing two-cell combinations were collected and studied.

FIG. 5A are time-lapse photographic images of the droplet sorter detecting and sorting cell pairs in droplets.

FIG. 5B is a bar graph showing the frequency of EGFP⁺ droplets following co-incubation of p65^{EGFP} reporter astrocytes with LPS-pre-stimulated or unstimulated macrophages 4-, 24- and 48-hours post-encapsulation.

FIG. 6A are droplet cytometry plots showing the gating strategy for unstimulated control. Left gate: EGFP+ cells. Right gate: paired cells in droplets.

FIG. 6B are droplet cytometry plots showing the gating strategy for non-targeting controls. Left gate: EGFP+ cells. Right gate: paired cells in droplets.

FIG. 6C are droplet cytometry plots showing the gating strategy for experimental macrophage conditions. Left gate: EGFP+ cells. Right gate: paired cells in droplets.

FIG. 6D are droplet cytometry plots showing the gating strategy for an unstimulated control. Left gate: EGFP+ cells. Right gate: paired cells in droplets.

FIG. 6E are droplet cytometry plots showing the gating strategy for non-targeting controls Left gate: EGFP+ cells. Right gate: paired cells in droplets.

FIG. 6F are droplet cytometry plots showing the gating strategy for experimental=microglia conditions at 24-hours post-encapsulation. Left gate: EGFP+ cells. Right gate: paired cells in droplets.

FIG. 6G is a bar graph quantifying the relative activation of reporter cells in droplets containing cell pairs that consist of macrophages or microglia together with astrocytes.

FIG. 6H is a bar graph showing analysis of anti-inflammatory pathways differentially activated in EGFP+ versus EGFP-fractions of p65^{EGFP} reporter astrocytes co-encapsulated with a perturbed microglial cell.

FIG. 6I is a table and schematic showing predicted upstream regulators and their transcriptional modules differentially expressed in EGFP+ versus EGFP-fractions of p65^{EGFP} reporter astrocytes co-encapsulated with a perturbed microglial cell.

FIG. 6J is a scatterplot of the number of guides detected and the number of drops sorted for SPEACC-seq experiments. n=9 experiments.

FIG. 6K is a graph showing the quantification of fold enrichment of non-targeting sgRNAs detected in each of the EGFP+ or EGFP-fractions of p65^{EGFP} reporter astrocytes co-encapsulated with a perturbed microglial cell. n=17-26 sgRNAs per group. Unpaired two-tailed t-test.

FIG. 7A is a schematic diagram showing the initial steps of the workflow of Example 2.

FIG. 7B is a schematic diagram showing how droplet collection, genomic DNA extraction, and sgRNA recovery via PCR was used to generate a library for sequencing.

FIG. 7C are graphs showing the results of analysis of sgRNA guides detected (left) in the genomic DNA of microglia from sorted droplets containing an EGFP+ astrocyte (middle). SPEAC-seq hits were filtered against an RNA-seq database of LPS-activated primary mouse microglia (right). The volcano plot represents expression of LPS-treatment relative to vehicle-treatment, n=3 per group.

FIGS. 7D-7F are a series of graphs and schematics that represent droplet CRISPR screens with p65^{EGFP} reporter astrocytes. In each of the figures, the left-side shows identification of genes in positive (EGFP+) droplets; the middle is a schematic of the stimulation and pairing scheme; and the right is a gene ontology analysis of hits identified in each screen. p65^{EGFP} reporter astrocytes were incubated under the following conditions. Partner cells were transduced with a genome-wide CRISPR/Cas9 lentiviral library: (FIG. 7D) stimulated bone marrow-derived macrophages (24-hr of 100 ng/mL LPS-EB); (FIG. 7E) stimulated (24 hr of 100 ng/mL TNFα/IL-1β) astrocytes; (FIG. 7F) stimulated bone marrow-derived macrophages (24-hr of 100 ng/mL LPS-EB).

FIG. 8A is a Venn diagram depicting the overlap of filtered SPEACC-seq hits filtered based on RNA expression from FIG. 7C with 4 independent bulk or single-cell RNA-seq datasets of microglia that were previously published (27, 34, 35, 78).

FIG. 8B is a bra graph of results of an analysis of gene expression for the candidate factors Areg, Fgl1, Pnoc, and Nrtn obtained by Guttenplan et al., in (78) (n=3 per group)

FIG. 8C is a bra graph of results of an analysis of gene expression for the candidate factors Areg, Fgl1, Pnoc, and Nrtn obtained in Example 2 (n=4 per group).

FIG. 8D is a bar graph of pathways detected by SPEAC-seq that limit astrocyte NF-κB activation discovered through bioinformatic analysis.

FIG. 8E is a pie chart of an analysis of secreted signals perturbed in microglia enriched in SPEAC-seq data and a table of the secreted molecules.

FIG. 9A is a schematic diagram that shows the construction of a barcoded lentiviral library for in vivo Perturb-seq analysis of candidate astrocyte receptors.

FIG. 9B is a UMAP plot of astrocytes captured by Perturb-seq from n=4 EAE mice.

FIG. 9C is a heatmap showing the analysis of NF-κB signaling activation as a function of Perturb-seq-based knockdown of candidate astrocyte receptors.

FIG. 9D is a schematic diagram that shows Qiagen IPA network analysis showing that EGFR signaling limits TNFα and IL-1β-driven NF-κB signals. Right-tailed Fisher's exact test.

FIG. 9E is a pair of graphs that show Egfr and Areg expression determined by qPCR in primary astrocytes and microglia from naïve or EAE mice. n=5 per group. Unpaired two-tailed t-test.

FIG. 9F is a heatmap that shows analysis of the transcriptional effects of AREG in human astrocytes pre-treated with pro-inflammatory cytokines and recombinant AREG. n=3 per group.

FIG. 9G is a graph that shows EAE disease course in mice transduced with Itgam::Cas9 lentiviruses co-expressing sgAreg or sgScrmbl. n=14 sgScrmbl, n=12 sgAreg mice. The experiment was repeated three times. Two-way repeated measures ANOVA.

### DETAILED DESCRIPTION

Provided herein are high-throughput systems and methods for identifying and understanding interactions between cells and/or molecules.

### Platform/Method for Studying Interactions Between Particles

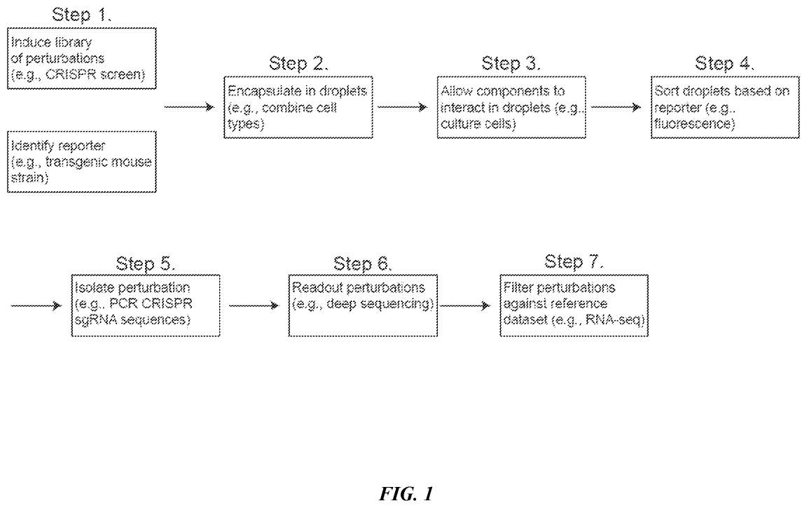

FIG. 1 illustrates the main steps in Systematic Perturbation of Encapsulated Associated Cells followed by Sequencing (SPEAC-seq) protocol.

### A. Partner Particles

a. Induce a Library of Perturbations/Variants in a First Particle Type

In step one of the SPEAC-seq protocol, to identify and/or study interactions mediated by different mechanisms, a population of a first type of particles, such as cells or phage, is combined with a library of interest. Particles are transduced (that is, combined) with the library of interest by routine methods known in the art and are dependent on the library of interest. The library of interest can include any genetic perturbation library (e.g., a CRISPR/Cas9 library, an RNAi library), a phage display library, a library of chemically diverse small molecules, a library of mutant versions of a protein, or a library of gene promoters or enhancers.

In the case of a CRISPR/Cas9 library, libraries are made by generating gRNAs targeting genes (see, e.g., J. G. Doench et al., Optimized sgRNA design to maximize activity and minimize off-target effects of CRISPR-Cas9. Nat Biotechnol 34, 184-191 (2016)). The library is then amplified by transformation of electrocompetent cells (e.g., STBL4, or the like), grown, and purified. Lentivirus containing the CRISPR/Cas9 library is then produced (see, e.g., D. Pan et al., A major chromatin regulator determines resistance of tumor cells to T cell-mediated killing. Science 359, 770-775 (2018)). Briefly, the pooled library is co-transfected with packaging plasmids into suitable cells (e.g., HEK293T cells). After a suitable time after transfection, lentiviral media is collected. Cells (e.g., microglia) are then transduced at an appropriate MOI (multiplicity of infection).

In the case of a phage display library, libraries are made by expressing a diverse set of peptides as fusions to bacteriophage coat proteins. The phage library is generated by standard cloning and production of phage particles.

In the case of a library of chemically diverse small molecules, libraries can be made using several methods including by creating DNA encoded chemical libraries, by co-encapsulating chemicals with a DNA- or RNA-barcode that uniquely defines their identity, or by co-encapsulating chemicals with a fluorescent barcode. In each case, the nucleic acid or fluorescent barcode is later used to identify the identity of the chemical. In this case, the library of chemically diverse small molecules can be delivered by any suitable means (e.g., lipid comprising components, such as lipid nanoparticles, etc.).

In the case of a library of mutant versions of a protein, libraries can be generated by random mutagenesis, by semi-random approaches, or can be computationally synthesized. Protein libraries can be made by modifying a cell's genome or introduced using a gene delivery system.

In the case of a library of gene promoters or enhancers, the intended elements can be cloned into expression vectors and introduced into cells using transfection or transduction.

b. Identify a Reporter in a Second Particle Type

A second particle type that, under normal biological conditions, responds (i.e., is activated or inhibited) to cues from the first particle type, contains a reporter that can indicate the state of a response (i.e., activation or inhibition). Typically, the reporter can be easily and rapidly measured by optical means (e.g., fluorescent reporter molecules, such as, GFP, YFP, RFP). The reporter may be genetically encoded, an enzymatic assay, or any marker that can be measured by antibody staining, DNA or RNA hybridization. However, the reporter may also be an image-based readout, or any property that can be used to sort or select for pairs of interest. The reporter can be coupled to multiple readouts including transcriptional activation or inhibition, soluble factor release or lack thereof, ligand-receptor binding or lack thereof, or any other cellular state such as metabolic status, ionic activity, or organellar reorganization such as phagocytosis, that is coupled to an optical readout of that state.

In the case of studying genetic perturbations (e.g., CRISPR/Cas9 perturbations), a second particle type can be a cell that expresses or contains a reporter molecule (e.g., a cell that expresses GFP).

In the case of a phage display library, the first particle type can be a bacteriophage that presents fragments of a peptide, antibody, scFabs, nanobodies, or receptor libraries on the cell surface and the second particle type can be a reporter that is a cell expressing a B cell receptor coupled to activation of a reporter molecule (e.g., GFP).

In the case of a library of chemically diverse small molecules, the first particle type can be a cell that expresses or contains a small molecule together with an expressed nucleotide barcode sequence that is captured by deep sequencing (see, e.g., the section “Droplet Breaking, Isolation of Perturbation, and Downstream Analysis”) and the second particle type can be a reporter that is a partner cell expressing a defined signaling cascade coupled to activation of a reporter molecule (e.g., GFP).

In the case of a library of mutant versions of a protein, the first particle type can be a cell expresses or contains a cDNA open reading frame encoding a protein and the second particle type can be a reporter that is a partner cell expressing a defined signaling cascade coupled to activation of a reporter molecule (e.g., GFP).

In the case of a library of gene promoters or enhancers, the first particle type can be a cell that expresses or contains a DNA sequence derived from a promoter or enhancer region and the second particle type can be a reporter that is a partner cell expressing a defined signaling cascade coupled to activation of a reporter molecule (e.g., GFP).

In each of the applications noted above (except for small molecule libraries) the reporter can be introduced into the second particle type through transfection/transduction means or any routine methods known in the art. In the case of a small molecule library, the reporter can be introduced into the second particle type using, e.g., lipid nanoparticles or other suitable means for delivery of the small molecule.

### B. Cell Encapsulation in Droplets

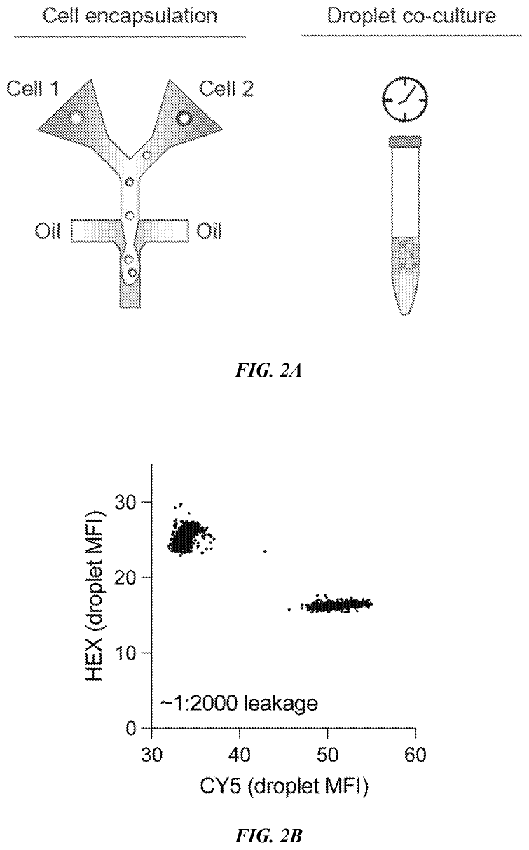

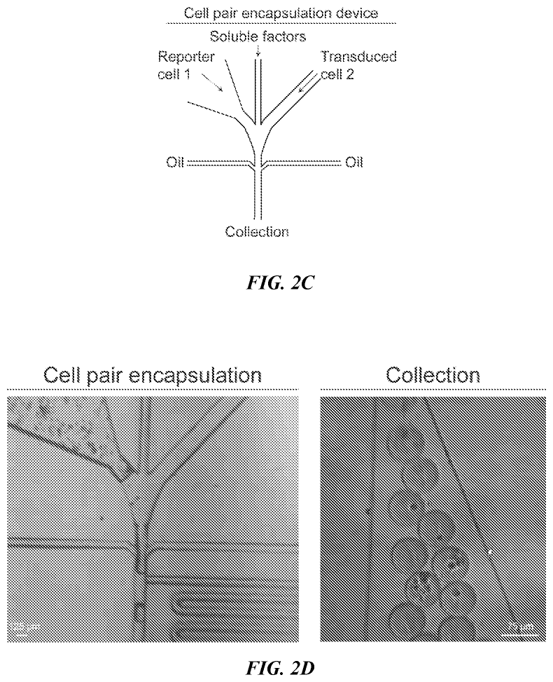

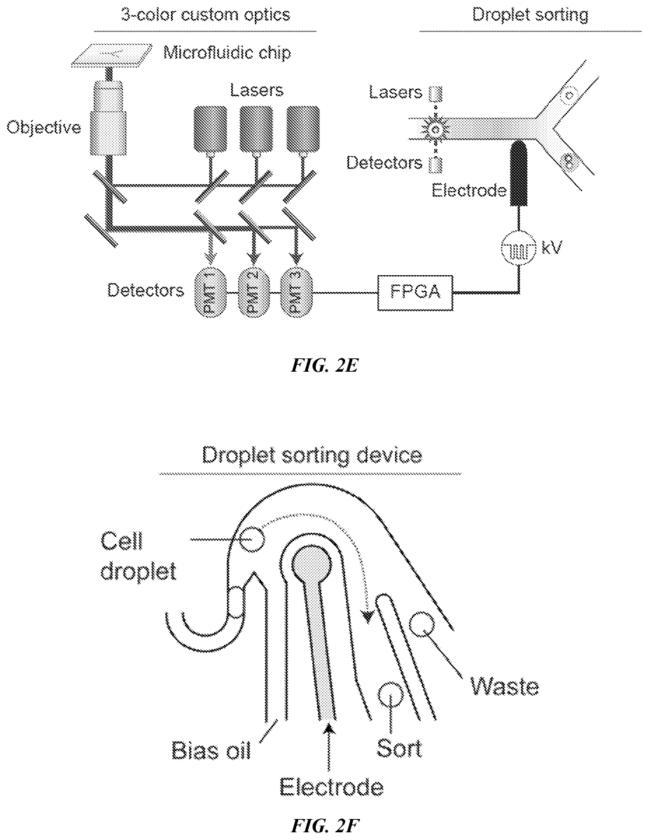

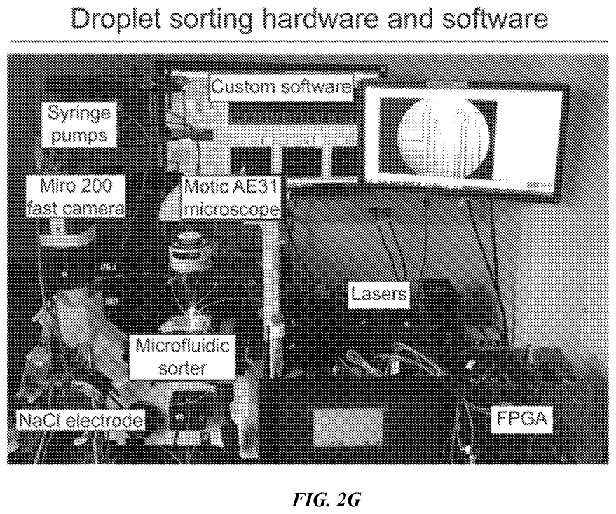

Once the two types of particles are prepared, they are encapsulated into picoliter or nanoliter water-in-oil droplets, as shown in FIG. 2A (left) and FIG. 2C, with the goal of obtaining at least some droplets that contain one of each type of particle. While the present invention is not limited to just cells, FIG. 2 shows one example in which the particle of type 1 and the particle of type 2 are cells. As shown in FIGS. 2A and 2C, cells of type 1 are carried through a microfluidic channel on the left, and cells of type 2 flow through a microfluidic channel on the right to a point where the two channels combine into one channel, and the one channel is then joined by two separate channels that provide a non-aqueous fluid, such as an oil, that passes through a nozzle or pinch-point that generates droplets of the aqueous carrier fluid for the cells within the non-aqueous fluid for collection. Soluble factors, if any, can be provided through another microfluidic channel upstream of the location where the droplets are formed.

Microfluidic encapsulation systems are known in the art and any suitable means for encapsulation can be used. In general, encapsulation of one each of the two types of cells can be achieved using Poisson statistics analysis to determine the proper concentration of each type of cell in its respective carrier fluid. For example, modulating concentration of each cell type is one method for achieving this isolation. In the present case, the concentration of each cell in its respective suspension prior to encapsulation is dependent on the anticipated droplet size. For instance, the cells comprising the reporter are overloaded into the system whereas the cells comprising the library of interest are at a diluted concentration. The rationale for doing so is to prevent droplets containing more than one cell with a perturbation. Reporter cells were loaded at a higher concentration, sometimes resulting in more than one reporter cell to enter a droplet to increase the probability of paired cells within droplets. The reporter cells were loaded at roughly 6.95*10\hat{ }6 cells/mL and the perturbed cells were loaded at roughly 1.39*10\hat{ }6 cells/mL (5-fold lower concentration than the reporter cells). Once the required concentrations of cells in each suspension has been generated, the suspensions are injected into a microfluidic device. Water-in-oil droplets can be generated using a droplet making instrument, e.g., a droplet generator such as the Biorad Automated Droplet Generator.

Other systems for encapsulation can also be utilized, such as the system described in U.S. Pat. No. 9,068,181, which is incorporated herein by reference in its entirety. Briefly, particles (e.g., the two partner cells), in two separate fluid streams can be encapsulated in individual droplets by first forming an ordered stream of particles in the fluid stream within each microchannel and then combining the two fluid streams containing ordered streams of particles into droplets each containing one of each of the two types of cells. Of course, there will be many droplets that are empty, and many droplets that contain only one type of cell.

### C. Droplet Co-Incubation

In step three (FIG. 1), and as shown in FIG. 2A (right image), the droplets are then incubated to allow interaction of the particles/components within the droplets, e.g., in one or more vessels, such as plastic or glass tubes, flasks, etc. This can be done at typical cell culture conditions (e.g., 37° C., 5% CO₂ for 10-72 hours). The conditions of this droplet co-incubation step can be modified based on the types of particles present within the droplet, and would be readily apparent to one of skill in the art (e.g., in the situation of a phage library).

### D. Droplet Sorting

Once again, the present invention envisions various particles. The Examples and Figures show one example in which the particles are cells. In step four (FIG. 1), after incubation of particles-containing droplets, the emulsion containing the droplets is injected into a droplet sorter to sort out the droplets that contain both the first and second cells. Droplet sorting by any means suitable in the art, for example, as described in Clark et al., “Concentric electrodes improve microfluidic droplet sorting,” Lab Chip, 18 (5): 710-713 (2018); Shields et al., “Microfluidic cell sorting: a review of the advances in the separation of cells from debulking to rare cell isolation,” Lab Chip, 15 (5): 1230-49 (2015); and Baret et al., “Fluorescence-activated droplet sorting (FADS): efficient microfluidic cell sorting based on enzymatic activity,” Lab Chip, 9 (13): 1850-8 (2009), the entireties of which are hereby incorporated by reference. Other options for droplet sorting include, but are not limited to, electromagnetism, fluid flow forces, or physical partitions. In some embodiments, a combination of different sorting methods can be used.

In some embodiments of the present invention, droplets are sorted based on three fluorescent criteria: the size of the droplet, the presence of an activated reporter, and finally the presence of the two cell particle types. This is then followed by a dielectrophoretic sorting. More particularly, as shown in the diagrams of FIGS. 2E and 2F, the droplets are detected and sorted using a dielectrophoretic microfluidic sorter system that includes an objective, three lasers that illuminate the cells at three different wavelengths, e.g., three detectors, one for each color, custom two, three, four (or more)-color optics and a programmed FPGA that activates an electrode when the droplets carrying two different, activate cells pass by the laser detector to be sorted into a sort/collection channel, whereas all empty droplets and those that contain only one cell are directed to a waste channel (Clark et al., supra 2018).

In particular embodiments, the system allows for a specific gating to be implemented such that the system allows the identification of droplets with a reference dye, and the presence of cells located within those droplets (that is, each cell type has its own cell dye), and the fluorescence of the reporter within one of the reporter cells. For example, in some embodiments, a reference dye at a low concentration is used to mark and identify droplets as they pass through the laser. This serves two important purposes. First, it allows for gating based on drop size, which is used to remove (by gating) coalesced drops. Second, it unambiguously determines which cells are co-located (paired) within a drop. Before encapsulation, cells are uniquely dyed with a cell viability stain. Stained cells are recorded as strong fluorescent puncta within a droplet, which allows their viability and position within the drop to be determined. Because of this spatial resolution, it is possible to distinguish between the fluorescence emitted by each cell within a drop individually, and clearly gate reporter cells with the desired fluorescence properties.

FIG. 2G shows one example of an overall SPEAC-seq system including syringe pumps to flow cells in their aqueous carrier fluids through a microfluidic droplet generator, a fast camera (e.g., Miro 200) to view the droplets in the microfluidic channels of the microfluidic droplet generator, a salt (NaCl) electrode, a microfluidic sorting system, a microscope (e.g., Motic AE31), lasers, a programmed FPGA, and a computer system with software to run the various systems

### E. Droplet Breaking, Isolation of Perturbation, and Downstream Analysis

In step 5 (FIG. 1), the droplets are broken by any suitable means, such as by using a chemical (e.g., perfluorooctanol (PFO)), by applying an electric field to break the droplets, or by thermal fluctuations. In some embodiments, the droplets are broken by first undergoing a few freeze-thaw cycles and then treated with a PFO containing reagent. The breaking procedure releases the drop contents so that they can be recovered for downstream analysis. Then one isolates the perturbation. In the case of a CRISPR/Cas9 library, once the droplets are broken, DNA containing the CRISPR sgRNA sequences is amplified (e.g., by PCR) for downstream analysis.

In the other applications envisioned by the present application, DNA, RNA, or protein would be isolated from broken droplets which would enable the identification of other macromolecule libraries introduced into droplets. Additionally, cells within droplets could be uniquely barcoded through the use of antibody tags against surface molecules, lipids, or other cellular components to enable single-cell processing of sorted droplets. Alternatively, droplets sorted into a well-plate would enable single-cell processing.

### F. Readout of Perturbations/Variants

In step 6 (see, e.g., FIG. 1), the perturbations are read out. This is dependent on the study that is being performed. Suitable downstream analysis include the following: deep sequencing methods, any activity assays that accesses biological readouts, affinity assays, protein binding assays, mass spectrometry, or further cell culture.

For example, when a CRISPR/Cas9 library is being studied, deep sequencing allows for the analysis of the sgRNA sequences. In the other applications envisioned by the present application, deep sequencing allows for the analysis of DNA sequences from enhancer regions, the sequence of constructs on which mutagenesis was performed, the nucleotide barcode co-delivered with a small molecule, or phage display DNA library sequences.

### G. Filter Perturbations

In step seven (FIG. 1), the perturbations are filtered against reference datasets to prioritize them for further analysis. For example, if the screen uses CRISPR/Cas9 perturbations and generates a list of sgRNA that are enriched in microglia that activate or inhibit an astrocyte inflammatory state, a microglia RNA-seq dataset would be used. sgRNA hits in genes that are known to be expressed the microglia RNA-seq dataset would be selected. For other applications envisioned, candidate hits would be screened in downstream assays that are well known in the art, including gene or protein expression profiling, heterologous expression systems, or functional validation.

### Applications for Using the Platform

### A. Genetic Library (e.g., CRISPR/Cas9 Library)

Cell-cell interactions throughout the body play important roles in various types of diseases. In many cases, however, little is known about the specific molecular pathways involved, and methods for their systematic identification are limited. To identify key molecules that are involved in specific signaling pathways in a given cell-type, a genetic library (such as a CRISPR/Cas9 library as described above) can be stably transduced in a first type of particles, i.e., microglial cells. In this situation, the second particle type (i.e., astrocyte cells) are transduced with a fluorescent reporter that can be used to identify cell pairs that form functional interactions. Astrocytes and microglial cells are known to interact with one another. The presence or absence of fluorescence provides a means to identify the molecular mechanisms, including signaling pathways, which mediate this interaction.

Once the first and second particle types (i.e., the first and second cell types) are generated, a single one of each type of cell is encapsulated into droplets. The droplets are then co-incubated to allow interaction of the cells to occur. The droplets are then sorted based on the reporter (e.g., fluorescence). Once the droplets are sorted, they are broken and the perturbations (e.g., variants included in the library) are isolated. In the case of a genetic library (such as a CRISPR/Cas9 library), the perturbations are PCR amplified and the perturbations (e.g., sgRNA sequences) are sequenced (e.g., deep sequencing). The sequences can then be further analyzed by comparing the perturbations/variants again a reference database.

### B. Screening Antibodies

Using a phage display peptide library expressed in bacteriophages, binding of displayed peptides to a specific B cell clone expressing a known B cell receptor (BCR) would trigger a BCR transcriptional response that would induce a fluorescent signal. Bacteriophages expressing the diverse peptide library would then be co-encapsulated using SPEAC-seq microfluidics with a B cell of interest. The extracellular activation signal would lead to fluorescence by activating downstream gene expression that triggers a fluorescent protein, or alternatively peptide binding blocks a fluorescent signal. In the first case, droplets would be sorted by fluorescence and in the second, droplets would be sorted by a lack of fluorescence. This strategy would identify peptide-antibody affinities.

### C. Screening Small Molecule Libraries

A library of chemically diverse small molecules, each co-encapsulated or tagged with a DNA barcode, would be loaded into a cell line such as HEK293 cells through nanoparticle transfection. HEK293 cells could secrete these small molecules to trigger signaling cascades in a partner cell whose responses are of experimental interest (such as primary immune cells, iPSCs, brain cells such as glia or neurons, or cells from other tissues) that fluoresces upon activation. Alternatively, the molecule activity could inhibit a constant fluorescent signal (such as the expression of a gene that marks a differentiation state, for example Foxp3 in regulatory T cells or parvalbumin in interneurons). Droplets would be sorted based on activation or inhibition of the fluorescent signal respectively, and a nucleotide barcode co-administered with each small molecule could be isolated to determine the identity of the molecule.

### D. Directed Evolution

Cells, such as neurons, would each express one copy of a gene derived from a gene expression library encoding a single protein (e.g., GCaMP). In the library, each protein would express a slightly different protein encoded by a nucleotide sequence generated by error-prone PCR or alanine scanning. Neurons expressing one copy of each variant would be encapsulated with a partner cell, such as another neuron or a glial cell that transmits a cue to activate or inhibit the molecule that might be coupled to a fluorescent signal upon activation (e.g., calcium in the case of GCaMP, or dopamine in the context of dLight1). In this way, droplets showing fluorescence could be isolated and the DNA sequences of each mutant variant would be sequenced and isolated for further testing.

### E. Enhancer Elements

Libraries of gene promoters or enhancers would be transfected into cells such as brain cells including neurons or glia, that are coupled to the expression of a fluorescent signal such as enhanced green fluorescent protein (EGFP). A partner brain cell, such as neurons or glia, would be co-encapsulated within droplets and made to secrete effector molecules, such as by stimulation with exogenous cytokines or neurotransmitters, that activate these promoters or enhancers to screen for specific promoter or enhancer sequences that are most efficiently driven by a specific exogenous cue. Droplets would be sorted by fluorescence and the promoter/enhancer elements sequenced.

### F. Function-Blocking Antibodies

Cells, such as brain cells or immune cells, would be stained with a library of antibodies fused to DNA barcodes to uniquely identify them (first particle type). The library of barcoded antibodies can contain, for example, function blocking antibodies, antibodies targeting soluble factors, antibodies blocking receptors, or antibodies blocking ligand-receptor binding. A partner cell (second particle type), such as neurons or glia or immune cells, expressing a fluorescent reporter of a cellular state such as EGFP, would be co-encapsulated with the antibody-stained cell within droplets. Droplets containing fluorescent activation or inhibition would be sorted and the antibody barcode that is coupled to a given antibody would be analyzed by deep sequencing. This approach would enable the screening of function-blocking antibodies and their effect on cell-cell communication.

### EXAMPLES

The invention is further described in the following examples, which do not limit the scope of the invention described in the claims.

Multiple sclerosis (MS) is a chronic inflammatory disorder of the central nervous system (CNS) (1). Interactions among CNS-resident glial cells contribute to the pathogenesis of several neurologic diseases, including MS and its pre-clinical model experimental autoimmune encephalomyelitis (EAE) (2-13). Characterizing astrocyte-microglia interactions has the potential to identify candidate therapeutic targets for neurologic disorders. However, current methods do not causally link cellular cross-talk with molecular states (12, 14-16) and show a limited ability to detect transient cell-cell interactions mediated by surface or secreted factors. The following example show how the present platform can be utilized to understand how molecules interact with each other.

### Example 1: Development of a Droplet-Based Forward Genetic Cell-Cell Interaction Screening Platform

### Materials and Methods

A. Primary Astrocyte and Microglial Cultures from Neonatal Mice:

Procedures were performed as described previously (74). Brains of mice aged P0-P3 were dissected into PBS on ice. Cortices were discarded and the brain parenchyma were pooled, centrifuged at 500 g for 10 minutes at 4 C and resuspended in 0.25% Trypsin-EDTA (Thermo Fisher Scientific, #25200-072) at 37 C for 10 minutes. Trypsin was neutralized by adding DMEM/F12+GlutaMAX (Thermo Fisher Scientific, #10565018) supplemented with 10% FBS (Thermo Fisher Scientific, #10438026) and 1% penicillin/streptomycin (Thermo Fisher Scientific, #15140148), and cells were passed through a 70 μm cell strainer. Cells were centrifuged at 500 g for 10 minutes at 4 C, resuspended in DMEM/F12+GlutaMAX with 10% FBS/1% penicillin/streptomycin and cultured in T-75 flasks (Falcon, #353136) at 37 C in a humidified incubator with 5% CO₂, for 7-10 days until confluency was reached. Astrocytes were shaken for 30 minutes at 180 rpm, the supernatant was collected for microglia and the media was changed, then astrocytes were shaken for at least 2 hours at 220 rpm and the supernatant was aspirated and the media was changed again. Medium was replaced every 2-3 days. Compound treatment was performed for 2 hours with compounds diluted in DMEM/F12+GlutaMAX (Life Technologies, #10565042) that was supplemented with 10% FBS (Life Technologies, #10438026) and 1% penicillin/streptomycin (Life Technologies, #15140122). For serum free condition, astrocytes was cultured in N1 DMEM/F12 media as described previously (74). Compounds used in these studies are: 0.01 pg/mL-1 μg/mL IL-1β (R&D Systems, #401-ML-005, 100 μg/mL stock in PBS), 2 μg/mL puromycin (Invivogen, #ant-pr-1), 0.01 pg/mL-1 μg/mL TNFα (R&D Systems, #410-MT-010, 100 μg/mL stock in PBS), 100 ng/ml IL-6 (R&D Systems, #406-ML-005), 200 ng/mL GM-CSF (PeproTech, #315-03), 100 ng/mL LPS-EB (Invivogen, #tlrl-3pelps), 100 ng/ml recombinant mouse IL-33 peptide (R&D Systems, #3626-ML-010), 10 ng/ml AREG (R&D Systems, #989-AR-100, 100 μg/mL stock in PBS). For analysis of cell activation in droplets, compounds were diluted to 2× of their intended working concentrations and co-flowed with cell suspensions for droplet encapsulation. For pre-stimulation experiments, astrocytes were pre-treated with 0.1 pg/mL IL-1β/TNFα for 24-hours and subsequently stimulated with the indicated dose of GM-CSF or IL-6 for 24-hours.

B. Primary Microglia Culture from Adult Mice:

C57BL/6J mice at least 2 months of age were used. Prior to dissection, one to three mice were anesthetized by isoflurane. Brains were aseptically dissected into 10 ml of enzyme digestion solution consisting of 75 μL Papain suspension (Worthington, #LS003126) diluted in enzyme stock solution (ESS) and equilibrated to 37° C. ESS consisted of 10 ml 10× EBSS (Sigma-Aldrich, #E7510), 2.4 ml 30% D (+)-Glucose (Sigma-Aldrich, #G8769), 5.2 ml 1 M NaHCO₃(VWR, #AAJ62495-AP), 200 μL 500 mM EDTA (Thermo Fisher Scientific, #155750 20), and 168.2 ml ddH2O, filter-sterilized through a 0.22 μm filter. Samples were shaken at 80 rpm for 30-40 minutes at 37° C. Enzymatic digestion was stopped by adding 1 ml of 10× Hi-Ovomucoid inhibitor solution and 20 μL 0.4% DNase (Worthington, #LS002007) diluted in 10 ml inhibitor stock solution (ISS). 10× Hi-Ovomucoid inhibitor stock solution contained 300 mg BSA (Sigma-Aldrich, #A8806), 300 mg Ovomucoid Trypsin Inhibitor (Worthington, #LS003086) diluted in 10 ml DPBS and filter sterilized using at 0.22 μm filter. ISS contained 50 ml 10× EBSS (Sigma-Aldrich, #E7510), 6 ml 30% D (+)-Glucose (Sigma-Aldrich, #G8769), 13 ml 1 M NaHCO₃(VWR, #AAJ62495-AP) diluted in 170.4 ml ddH2O and filter-sterilized through a 0.22 μm filter. Tissue was mechanically dissociated using a 5 ml serological pipette and filtered through at 70 μm cell strainer (Fisher Scientific, #22363548) into a fresh 50 ml conical. The mixed suspension was centrifuged at 500 g for 5 minutes and resuspended in 10 ml of 30% (v/v) Percoll solution (9 ml Percoll (GE Healthcare Biosciences, #17-544501), 3 ml 10× PBS, 18 ml ddH2O). Percoll suspension was centrifuged at 500 g for 25 minutes with no brakes. Supernatant was discarded and the cell pellet was washed with DPBS, centrifuged at 500 g for 5 minutes and resuspended in 20 ml prewarmed DMEM/F12 (Gibco, 31331-093) including 10% (v/v) FCS, 1% (v/v) PS (Life Technology, #15140122), and 13% (v/v) L292 conditioned medium and seeded into an uncoated T75 cell culture flask (Sarstedt, #831813002). The medium was changed twice a week until the cells reached 90% confluency (12-16 days). To separate the mixed glia culture containing both astrocytes and microglia in an approximatively 2:1 ratio, cells were detached by using 10 ml prewarmed Trypsin-EDTA 0.05% (Thermo Fisher Scientific, #25200-072). After 10-15 minutes incubation in Trypsin-EDTA at 37° C., cells were collected through a 70 μm cell strainer into a 15 ml Falcon tube (Thermo Fisher Scientific, #352196) and the reaction was stopped by adding 5 ml pure FCS. The single cell suspension containing both microglia and astrocytes was centrifuged at 300 g at 4° C. for 10 minutes. After washing the cells, the pellet was resuspended in 5 ml sterile DPBS and the cells were counted. CD11B microglia and CD11B⁻ astrocytes were separated by using magnetic microbeads attached to an anti-mouse CD11B antibody (Miltenyi Biotec, #130049601) according to the manufacturer's protocol. Following the MACS separation, cells were centrifuged at 300 g at 4° C. for 5 minutes and resuspended in 5 ml sterile DPBS.

### C. Microfluidic Device Fabrication:

Master molds for microfluidic device fabrication were fabricated at the Harvard Medical School Microfluidics/Microfabrication Core Facility using common photolithography techniques. Briefly, silicon wafers (University Wafer) were spin-coated with SU-8 photoresist (Kayaku Advanced Materials), patterned with a photomask using ultraviolet light, and baked following the manufacturer's instructions. PDMS devices were fabricated from master molds as follows: Curing agent and PDMS prepolymer (Momentive, #RTV615) were mixed 1:10 and degassed in a vacuum chamber. The PDMS mixture was poured onto the master mold, further degassed, and baked at 65° C. for 4 hours. The PDMS replica was punched with a 0.75 mm biopsy punch (Harris Uni-Core) and bonded to a glass slide (75×50×1.0 mm, Fisher Scientific, #12-550C) using an oxygen plasma bonder (Technics Plasma Etcher 500-II). The device was placed on a hot plate at 150° C. for 10 minutes and baked at 65° C. for 4 hours. Finally, channels were rendered hydrophobic by treatment with Aquapel (Aquapel Glass Treatment) for 5 min.

### D. Microfluidic Cell Encapsulation:

For microfluidic cell encapsulation, cells were detached via a 10-minute incubation in TrypLE at 37° C. Cells were washed once and stained with CellTrace Far Red Cell Proliferation Kit (Thermo Fisher Scientific, #C34564) at 1 μM or CellTrace Calcein Red-Orange, AM (Thermo Fisher Scientific, #C34851) at 2 μM for 25 minutes at 37° C. Cells were washed, counted and resuspended at the required density based on the anticipated droplet size in DMEM/F12 without phenol red (Thermo Fisher Scientific, #21041025)+10% (v/v) FCS and 18-20% (v/v) Opti-prep (Sigma-Aldrich, #D1556-250ML). For optimal droplet detection, Cy5-alkyne (Sigma-Aldrich, #777358) was added with a final concentration of 100 nM to the cell population stained with CellTrace Far Red Cell Proliferation Kit. The cell mixtures were loaded into a 3 ml syringe (BD Biosciences, #309657) with a 27-gauge needle (BD Biosciences. #305109) and connected to the microfluidic device using PTFE tubing (Scientific Commodities, #BB31695-PE/2). For co-encapsulation and in-drop-stimulation experiments, the cell suspensions were injected into the microfluidic device by using a syringe pump at a flow rate of 600 μl/hour. Drops were generated by flow focusing of the resulting stream with QX200 droplet generation oil for EvaGreen (BioRad, #1864006) at a flowrate of 3000 μl/hour. The resulting emulsion was collected in 3 ml syringes (BD, #148232A) for reinjection or in a 15 ml Falcon tube (Thermo Fisher Scientific, #352196) for cell viability assessment. The emulsion was incubated at 37° C. 5% CO₂ for 10-72 hours.

### E. Reinjection and Sorting of Droplets:

After incubation of cell-containing droplets, the emulsion was reinjected onto a custom droplet-sorter as described in (25). In brief, the emulsion containing monodisperse drops was re-injected into a microfluidic device by using a syringe pump (Harvard Apparatus, milliliter OEM syringe pump) at a flow rate of 200 μl/hour. Drops were spaced by injection of 3M Novec 7500 Engineered Fluid (HFE; 3M, #Novec 7500) at a flow rate of 300 μl/hour. Electrode and moat channels were loaded with 2M NaCl solution. Detection of droplet fluorescence was performed using a custom in-house 3-color droplet cytometer. Three lasers (473 nm, 532 nm, 638 nm) are aligned via dichroic mirrors and focused on the microfluidic device mounted on a microscope (Motic AE31). A custom Lab View (National Instruments) program was used to run a field programmable gate array (FPGA; National Instruments) to control photomultiplier tubes (PMT) (PMM01/PMM02, Thorlabs) for fluorescence detection. Based on a pre-determined fluorescence-threshold, positive drops were sorted, using a concentric electrode design, into a separate output channel by actuating electric pulses via a high-voltage amplifier (Trek). Negative and positive populations were collected in 15 ml Falcon tubes (Thermo Fisher Scientific, #352196) and the resulting oil-phase was overlaid with 200 μl DPBS (Thermo Fisher Scientific, #14190250). For optimal droplet recovery, the emulsion was frozen at −80° C. Peak droplet fluorescence values were recorded, exported, and analyzed in FlowJo.

### F. Droplet Stability Assessment:

To assess droplet stability, primary astrocytes were encapsulated at 1 cell in every 10 drops as described above. The cells were resuspended and encapsulated in the following combinations of medium±surfactant, 10% (v/v) FCS (Thermo Fisher Scientific, #10438026) and 18% (v/v) Opti-prep (Sigma-Aldrich, #D1556-250ML): (i) DMEM/F12 with phenol red (Thermo Fisher Scientific, #11320033), (ii) DMEM/F12 without phenol red (Thermo Fisher Scientific, #21041025), (iii) DMEM/F12 without phenol red+0.1% (w/v) bovine serum albumin (Sigma-Aldrich, #A32944), and (iv) DMEM/F12 without phenol red+0.1% (v/v)+Pluronic F-68 (Thermo Fisher Scientific, #24040032). Droplets were collected in 15 ml Falcon tubes (Thermo Fisher Scientific, #14190250) and droplet stability was assessed by imaging on a Leica DMi8 Inverted Microscope as the number of coalesced droplets after one day of incubation at 37° C. 5% CO₂. To assess the transport of soluble protein between drops, 1 μM Bovine Serum Albumin (BSA) conjugated with tetramethylrhodamine (Thermo Fisher Scientific, #A23016) or 1 μM BSA conjugated with Alexa Fluor 647 (Thermo Fisher Scientific, #A34785) were separately encapsulated in 65-micron droplets with DMEM/F12 without phenol red (Thermo Fisher Scientific, #21041025)+0.1% (v/v) Pluronic F-68 using droplet generation oil for EvaGreen (BioRad, #1864006). Drops were mixed, incubated for 24 hours, and imaged using an ECHO Revolve Fluorescence Microscope.

### G. Cell Recovery and Viability Assessment:

To quantify cell viability cells were stained with CellTrace Calcein Red-Orange (Thermo Fisher Scientific, #C34851) according to the manufacturer's protocol prior to encapsulation. After droplet encapsulation, the oil phase was aspirated and droplets were merged and broken using 20% (v/v) 1H,1H,2H,2H-perfluoro-1-octanol (PFO; Sigma-Aldrich, #370533) in HFE (3M, #Novec 7500) to recover the cells trapped inside droplets. After the PFO break, the aqueous phase was separated by centrifugation at 30 g for 30 seconds and transferred into a fresh tube. Cells were washed and live cells were quantified by FACS on a BD LSRFortessa. To acquire micrographs of cells post-encapsulation, 10 μL of the emulsion was collected in a cell counting chamber slide (Thermo Fisher Scientific, #C10228) and imaged on a Leica DMi8 inverted microscope.

### H. ELISA

Costar 96-well plates (Corning, #3690) were coated with capture antibodies diluted in 1×PBS: anti-mouse TNF-α capture (Invitrogen, #88-7324-88, 1:250), anti-mouse IL-1 beta capture (Invitrogen, #88-7013-88, 1:250), overnight at 4° C. Plates were washed 3 times with 0.05% Tween in 1× PBS (Boston BioProducts, #IBB-171X) and blocked with 1× Elispot diluent (eBioscience, #00-4202-56) for 1 h at room temperature. The standard curve was prepared from 1 ng ml 1 protein diluted in 1× Elispot diluent. Samples were undiluted. Plates were washed 3 times with 0.05% Tween in 1× PBS and samples and standard curve were added and incubated overnight at 4° C. The next day, plates were washed 3 times with 0.05% Tween in 1×PBS and incubated with detection antibodies diluted in 1× Elispot diluent: anti-mouse TNF-α detection (Invitrogen, #88-7324-88, 1:250), anti-mouse IL-1 beta detection (Invitrogen, #88-7013-88, 1:250), for 1 h at room temperature. Afterwards, plates were washed 3 times with 0.05% Tween in 1× PBS and incubated with Avidin-HRP diluted in 1× Elispot diluent (Invitrogen, #88-7324-88, 1:100) for 1 h at room temperature. Next, plates were washed 6 times with 0.05% Tween in 1×PBS and revealed using 1× TMB Substrate Solution (Invitrogen, #00-4201-56) for 15 minutes at room temperature. The reaction was stopped by KPL TBM Stop Solution (SeraCare, #5150-0021) and plates were read at 450 nm with 560 nm reference value on a GloMax Explorer Multimode Microplate Reader (Promega).

### Results and Discussion

To establish a droplet microfluidic platform for the study of cell-cell interactions (see, FIG. 2A, showing a schematic showing how cells are co-encapsulated using microfluidics inside picoliter water-in-oil droplets (left) and how the droplets are co-incubated (right)), we optimized the cell culture media to ensure droplet stability over time, and confirmed that soluble factors did not transfer between droplets in 24-hours (FIG. 2B). As shown in the scatterplot in FIG. 2B, approximately 1:2,000 droplets contained both dyes in phenol red-free media after 24 hours of culture at 37° C. Microfluidic co-flow of two aqueous suspensions (one per cell type) and oil (FIGS. 2C and 2D) was used to generate picoliter water-in-oil droplets containing cell pairs. FIG. 2C is a schematic showing the flow of the cells in the encapsulation device. FIG. 2D shows actual images of the co-flow encapsulating device. The droplets were then detected and sorted using custom three-color optics and a dielectrophoretic microfluidic sorter (25). Specifically, FIG. 2E shows a schematic of how co-incubated cell pairs are monitored based on their fluorescence using a 3-color droplet cytometric system and are then sorted with dielectrophoresis to isolate cell-cell pairs. FIG. 2F is a schematic of the concentric droplet sorter showing the location of reinjected droplets, bias oil, sort electrode, and outlet channels. FIG. 2G is a picture of the system including: a custom droplet cytometer and software for detecting, gating, and sorting drops containing cell pairs based on fluorescence.

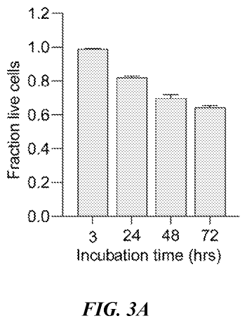

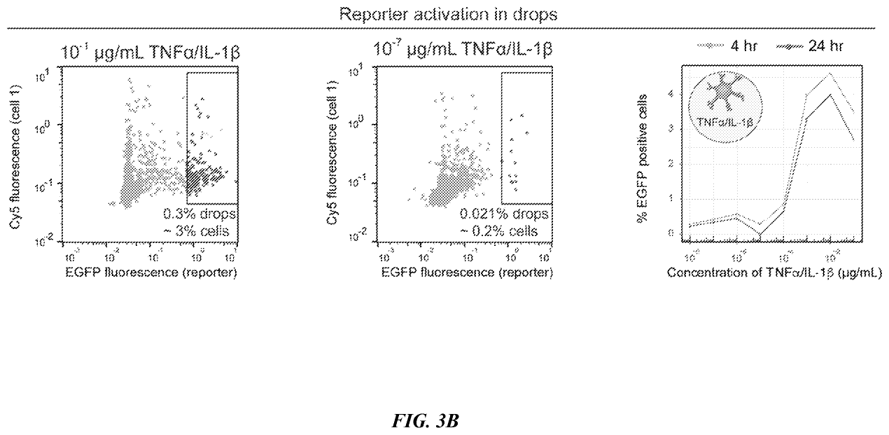

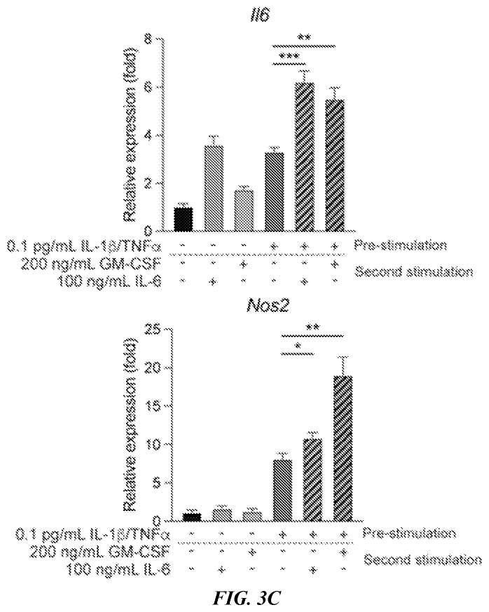

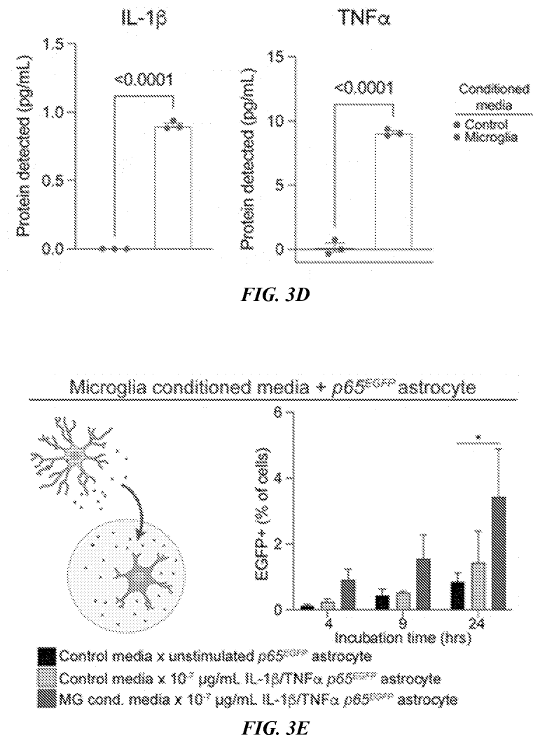

To validate this system, we first performed a time course analysis of cell survival by loading calcein-labelled cells into droplets and culturing them for 3-, 24-, 48-, or 72-hours at 37° C. As shown in FIG. 3A, we estimated cell survival rates of 95% and 80% by fluorescence activated cell sorting (FACS) and live/dead cell staining at 3- and 24-hours post-encapsulation in droplets, respectively, which significantly decreased at 48- and 72-hours post-encapsulation.

We then tested whether cells cultured in droplets responded to stimulation using transgenic astrocytes that express enhanced green fluorescent protein (EGFP) following nuclear factor kappa B (NF-κB) activation (26). Shown in FIG. 3B are graphs showing droplet cytometry of EGFP droplets following stimulation of p65^{EGFP} reporter astrocytes with 10⁻¹ μg/mL (left) or 10⁻⁷ μg/mL (middle) TNFα and IL-1β at 24 hours post-encapsulation. Droplet-encapsulated NF-κB reporter astrocytes displayed dose-dependent EGFP expression in response to co-encapsulation with increasing concentrations of the NF-κB-activating cytokines TNFα and IL-1β.

We next confirmed that astrocytes pre-stimulated with a sub-threshold dose of IL-1β and TNFα (0.1 pg/mL) were susceptible to subsequent activation with pro-inflammatory cytokines including IL-6 and granulocyte-macrophage colony-stimulating factor (GM-CSF), as expected (27). FIG. 3C shows that pro-inflammatory cytokine expression upon a 24-h pre-stimulation with a subthreshold dose of IL-1β/TNFα (0.1 pg/mL) with or without subsequent IL-6 or GM-CSF stimulation resulted in astrocyte activation (based on II6 expression, top graph and Nos2 expression, bottom graph). Indeed, as shown in FIG. 3D, we detected NF-κB activation in transgenic NF-κB reporter astrocytes cultured in droplets loaded with conditioned media from lipopolysaccharide (LPS)-activated microglia, which contained IL-1β and TNFα. Time course analyses detected EGFP expression 4-hours post-encapsulation of NF-κB reporter astrocytes with microglia conditioned media, with higher reporter activation detected 24-hours post-encapsulation (see, FIG. 3E, which shows frequency of EGFP droplets following co-incubation of sub-threshold stimulated or unstimulated p65^{EGFP} astrocytes with microglia conditioned media or control media at 4-, 9- and 24-hours post-encapsulation).

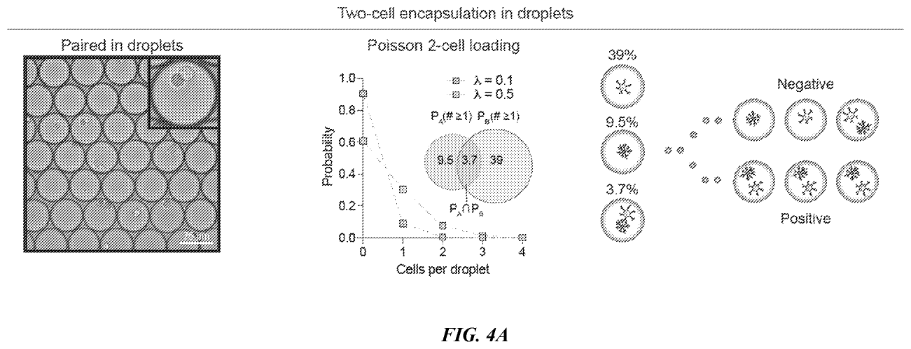

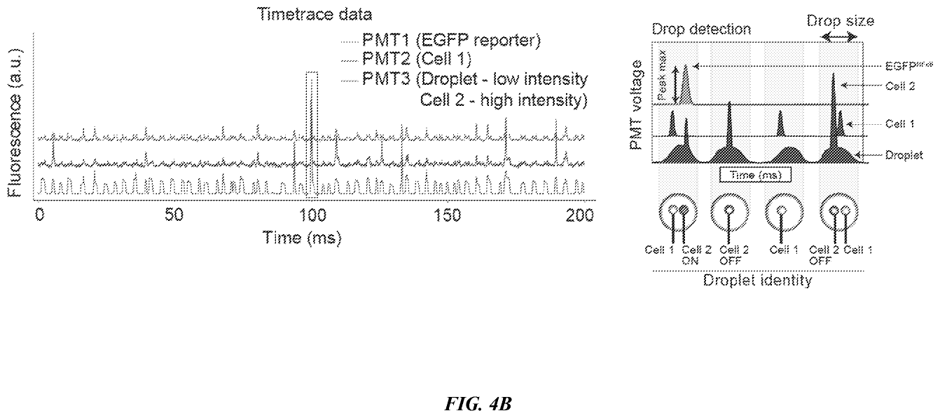

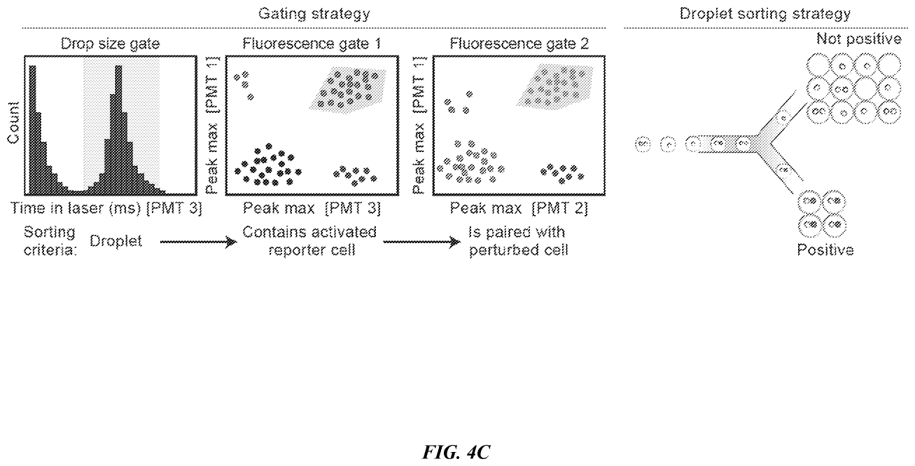

Finally, we extended our studies to cell pairs to determine whether cues produced by one cell were sufficient to alter the cellular state of a cell co-incubated in the same droplet. We developed and validated a system for the loading and detection of cell pairs in droplets using multiplexed labeling with cell permeant fluorescent dyes. Specifically, as shown in FIG. 4A, on the left is a micrograph showing cells co-incubated within droplets remain isolated from neighboring cell pairs and interact via direct contact and/or secreted soluble factors. Cell loading (middle graph and schematic on right) determines the probability that a drop contains each cell type. Next, we optimized droplet sorting parameters for the isolation of cell pairs displaying reporter activation. Specifically, as shown in, FIG. 4B droplet cytometric time trace data showed presence of droplet (photomultiplier tube 3 (PMT3), low sustained intensity), cell 2 (photomultiplier tube 3 (PMT 3), sharp intensity peak), EGFP reporter (photomultiplier tube 1 (PMT1)), and cell 1 (photomultiplier tube 2 ((PMT2)). An inert CY5 tracer dye was added to detect, and gate drops of the correct size (left). To sort the droplets the schematic shown in FIG. 4C was followed. The gating strategy showed how cell-cell pairs were identified by sequentially gating drops that 1) were the correct size, 2) contained an activated reporter cell (astrocyte), and 3) were paired with the desired cell-cell pair (astrocyte-microglia) and sorted such that only drops containing two-cell combinations were studied.

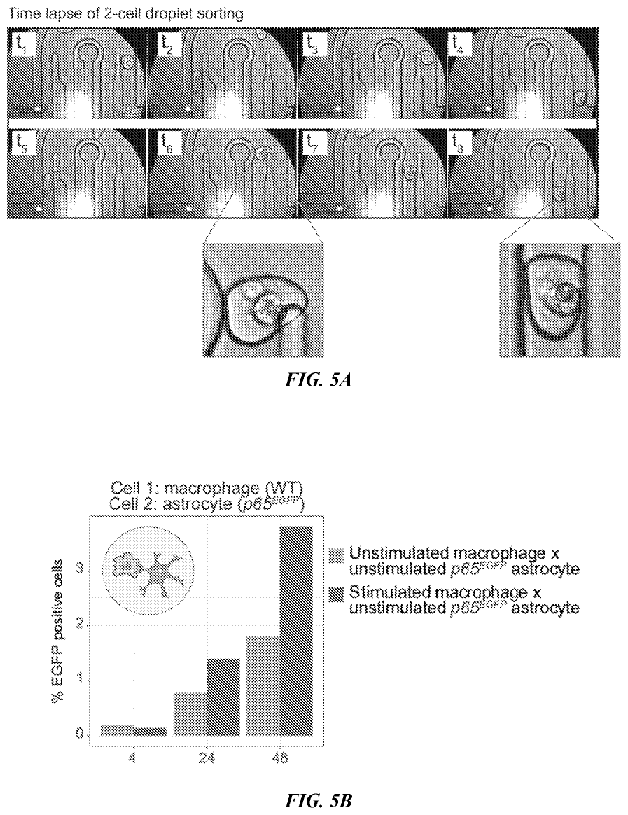

FIG. 5A shows time lapse images of the droplet sorter detecting and sorting cell pairs in droplets. Preliminary experiments detected the upregulation of EGFP expression in NF-κB reporter astrocytes co-encapsulated in droplets with activated, but not resting, macrophages. In FIG. 5B, the frequency of EGFP⁺ droplets following co-incubation of p65^{EGFP} FP reporter astrocytes with LPS-pre-stimulated or unstimulated macrophages 4-, 24- and 48-hours post-encapsulation is shown.

### Example 2: SPEAC-Seq Identifies Microglial Suppressors of NF-κB Signaling in Astrocytes

### Materials and Methods

### A. Genome-Scale CRISPR/Cas9 Library Production:

Amplification and sequencing of the plasmid library was performed as previously described (75). Briefly, a mouse CRISPR/Cas9 pooled lentiviral library consisting of 78,637 gRNAs targeting 19,674 mouse genes (29) (lentiCRISPRv2, Brie, Addgene #73632, a gift from David Root and John Doench) was obtained and amplified by transformation of STBL4 electrocompetent cells (Thermo Fisher Scientific, #11635018) according to the Broad Institute's Protocol: “Amplification of pDNA libraries”. After 16-18 hours of growth, pellets were collected and the library purified using an endofree plasmid maxi kit (Qiagen, #12362) according to the manufacturer's protocol with two modifications: a) add P1, P2, P3 directly to the conical and centrifuge to pellet lysed debris before adding to plunger; b) warm elution buffer to 50° C. before eluting. Lentivirus production was performed as previously described (21). The pooled library was co-transfected with packaging plasmids (psPAX2, Addgene #12260 and pCMV-VSV-G, Addgene #8454) into HEK293T cells using LT-1 transfection reagent (Mirus Cat #MIR2305) following the manufacturer's protocol. psPAX2 was a gift from Didier Trono (Addgene plasmid #12260; http://n2t.net/addgene: 12260; RRID: Addgene 12260). pCMV-VSV-G was a gift from Bob Weinberg (Addgene plasmid #8454; http://n2t.net/addgene: 8454; RRID: Addgene_8454) (76).

Library DNA (37 μg), psPAX2 DNA (46 μg) and VSV-G DNA (4.62 μg) was mixed and transfected into HEK293T cells in a T175 flask (Corning, #353112). Six hours after transfection, media was removed and replaced with 40 ml of virus production media (DMEM/F12 (Thermo Fisher Scientific, #11320033)+20% (v/v) FCS (Thermo Fisher Scientific, #10438026)). Forty-eight hours after transfection, lentiviral media was harvested and stored in −80 C. Cells were transduced at a MOI of 0.4, as described (75). In brief, cells were detached by using 10 ml prewarmed Trypsin-EDTA 0.05% (Thermo Fisher Scientific, #25200-072), counted and resuspended at 1.5×10⁶ cells/ml in growth medium supplemented with 16 μg/ml Polybrene (Millipore, #TR1003G) and the respective volume of lentiviral media.

B. Amplification and Analysis of sgRNA Sequences from Sorted Droplets:

After droplet encapsulation and sorting, the sgRNA target region was amplified and sequenced following the Broad Institute's protocol: “POR of sgRNAs from gDNA for Illumina Sequencing”. Sorted droplets were placed in the −80 C for a minimum of 24 hours, thawed at room temperature for 1 hour, and broken by adding 1 ml 20% (v/v) PFO in HFE (3M, #Novec 7500). For optimal phase separation, the emulsion was gently mixed by tapping the tube and subsequently centrifuged at 1000 g for 30 seconds. The aqueous layer containing the sorted cells was aspirated and transferred to a fresh 1.5 ml microcentrifuge tube. Genomic DNA (gDNA) was isolated using a Blood & Tissue DNA isolation kit (Qiagen, #69504) according to the manufacturer's protocol.

The genomic DNA was eluted in a final volume of 400 μl and subsequently concentrated to 40 μl by 2.0× AMPure XP bead purification according to the manufacturer's protocol (Beckman Coulter, #A63880). Next, the sgRNA libraries were amplified according to the Broad Institute's Protocol: “Amplification of pDNA libraries”. In brief, positive samples containing less than 2000 droplets were PCR amplified for 35 cycles, negative samples with more than 2000 droplets were PCR amplified for 28 cycles. A staggered forward primer cocktail, made by combining equimolar concentrations of P5 0 nt stagger 5′-

(SEQ ID NO: 1)
5′
ATGATACGGCGACCACCGAGATCTACACTCTTTCCCTACACGA
CGCTCTTCCGATCTGATGTCCACGAGGTCTCT-3′,
P5 1 nt stagger
(SEQ ID NO: 2)
5′-
AATGATACGGCGACCACCGAGATCTACACTCTTTCCCTACACG
ACGCTCTTCCGATCTCGATGTCCACGAGGTCTCT-3′,
P5 2 nt stagger
(SEQ ID NO: 3)
5′-
AATGATACGGCGACCACCGAGATCTACACTCTTTCCCTACACG
ACGCTCTTCCGATCTGCGATGTCCACGAGGTCTCT-3′,
P5 3 nt stagger
(SEQ ID NO: 4)
5′-
AATGATACGGCGACCACCGAGATCTACACTCTTTCCCTACACG
ACGCTCTTCCGATCTAGCGATGTCCACGAGGTCTCT-3′,
P5 4 nt stagger
(SEQ ID NO: 5)
5′-
AATGATACGGCGACCACCGAGATCTACACTCTTTCCCTACACG
ACGCTCTTCCGATCTCAACGATGTCCACGAGGTCTCT-3′,
P5 5 nt stagger
(SEQ ID NO: 6)
5′-
AATGATACGGCGACCACCGAGATCTACACTCTTTCCCTACACG
ACGCTCTTCCGATCTTGCACCGATGTCCACGAGGTCTCT-3′,
P5 6 nt stagger
(SEQ ID NO: 7)
5′-AA
TGATACGGCGACCACCGAGATCTACACTCTTTCCCTACACGAC
GCTCTTCCGATCTACGCAACGATGTCCACGAGGTCTCT-3′,
P5 7 nt stagger
(SEQ ID NO: 8)
5′-
AATGATACGGCGACCACCGAGATCTACACTCTTTCCCTACACG
ACGCTCTTCCGATCTGAAGACCCGATGTCCACGAGGTCTCT-3′,
P5 8 nt stagger
(SEQ ID NO: 9)
5′-5′-
AATGATACGGCGACCACCGAGATCTACACTCTTTCCCTACACG
ACGCTCTTCCGATCTGAAGACCCTTGTGGAAAGGACGAAACAC
CG-3′,
and a unique P7 primer for use with
lentiCRISPRv2:
(SEQ ID NO: 10)
5′CAAGCAGAAGACGGCATACGAGATNNNNNNNNGTGACTGGA
GTTCAGACGTGTGCTCTTCCGATCTCCAATTCCCACTCCTTTC
AAGACCT-3′

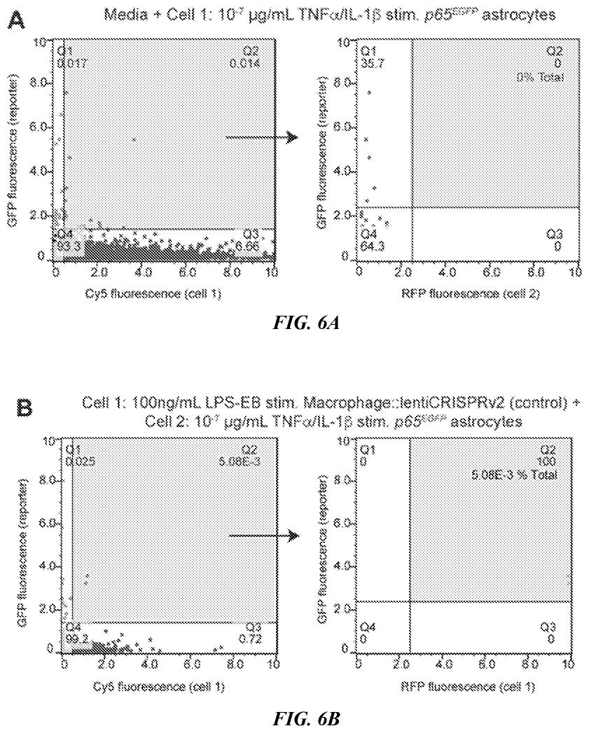

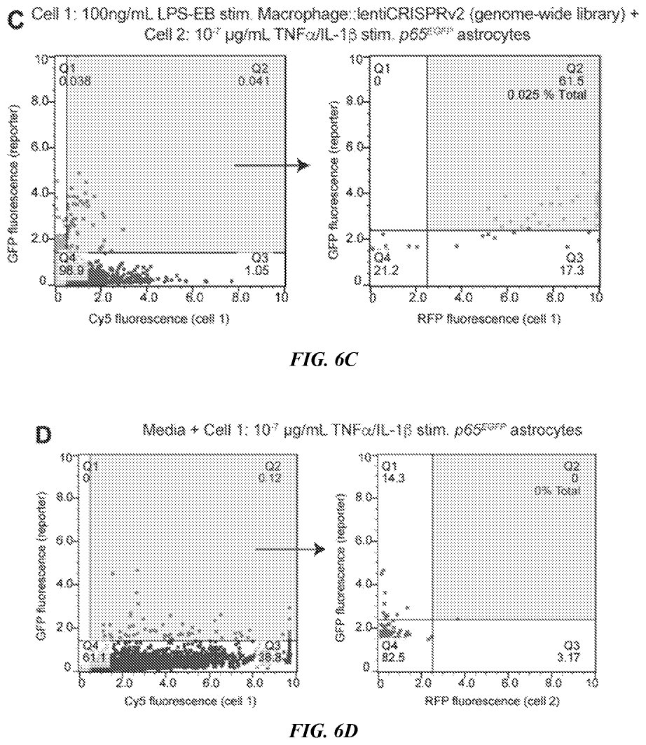

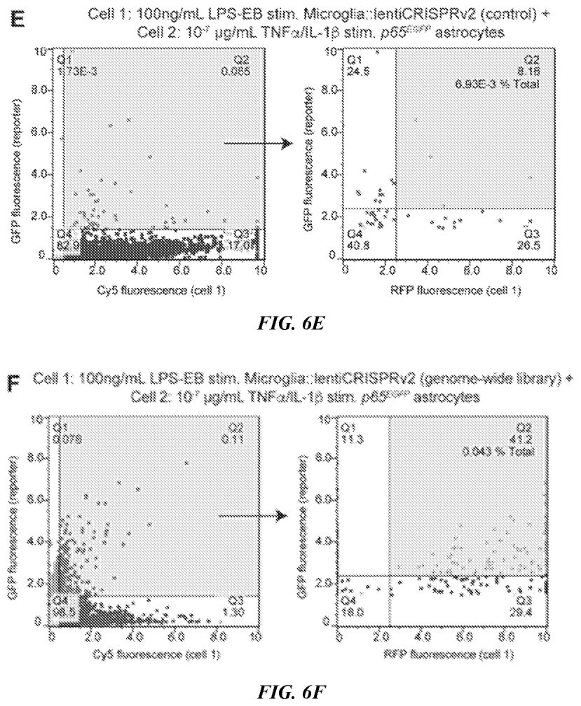

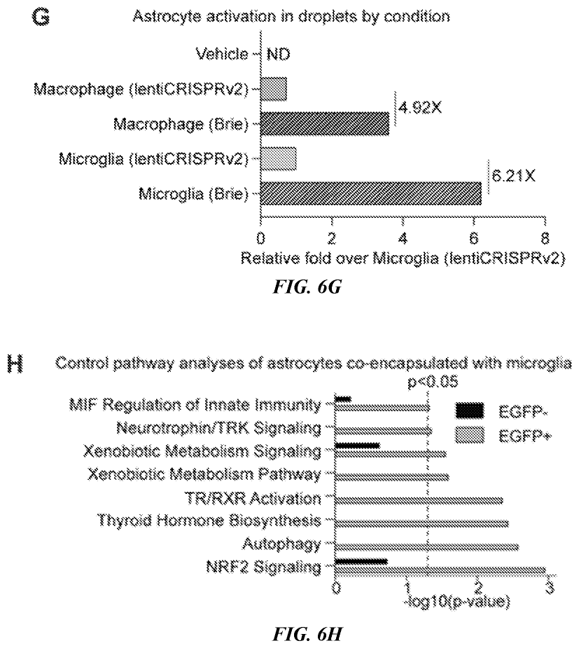

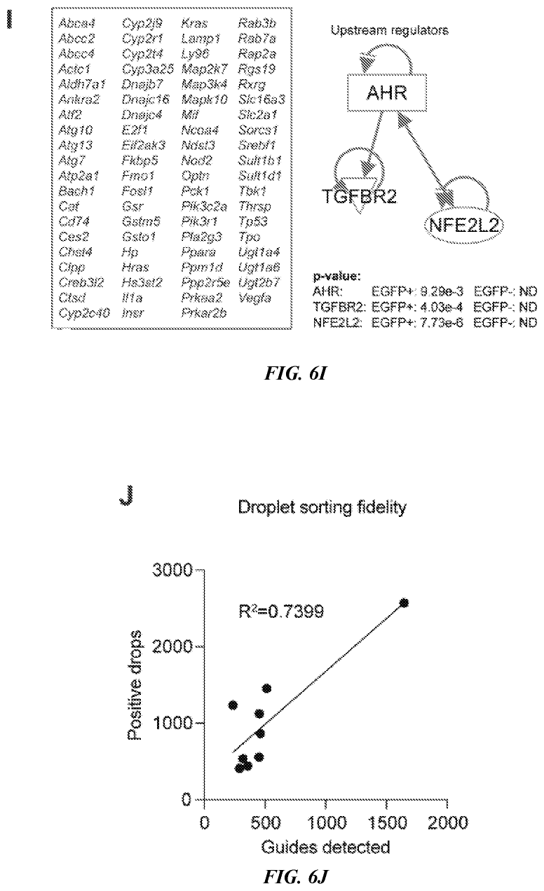

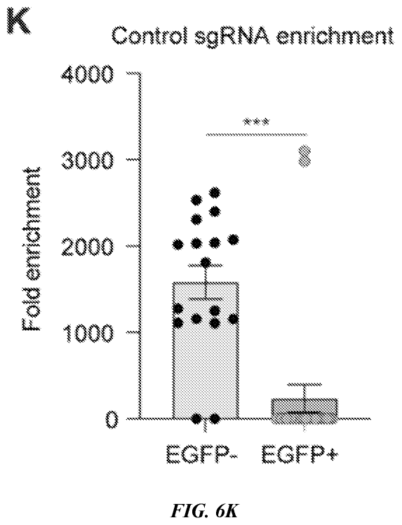

were used. Following library amplification, the samples were purified using 2.0× AMPure XP bead purification and eluted in 40 μl H2O. Finally, the sgRNA libraries were sized on an Agilent 2100 Bioanalyzer and their concentration determined using a KAPA Library Quantification Kit (Kapa Biosystems, #KK4824) according to the manufacturer's protocol. Libraries were sequenced at 1×75 bp at the Harvard Biopolymers Facility on a MiSeq Micro or in the Neurotechnology Studio of Brigham and Women's Hospital on a NextSeq550. Raw sequencing reads were trimmed using cutadapt to find sgRNA sequences matching those contained in the genome-wide library (-g ACACCG . . . GTTTTAG) and counted using MAGeCK (77). The non-targeting guides with high fold enrichment in FIG. 6K are control285 and control946 from the Brie library.

### C. Rationale and Criteria for Selecting Positive Hits:

To filter the hits detected by SPEAC-seq against physiologically relevant processes in microglia, we retained for analysis only SPEAC-seq genes detected in a filtered bulk RNA-seq dataset of primary microglia treated with 100 ng/mL of LPS-EB (Invivogen, #tlrl-3pelps) or vehicle. In addition, we validated this filtered list against four previously published mouse bulk and single-cell RNA-seq studies that analyzed microglia gene expression in vivo in health and disease (27, 34, 35, 78).

### Results and Discussion

We combined the droplet-based co-culture system with CRISPR/Cas9 perturbations to establish SPEAC-seq, a platform for forward genetic screens of regulatory cell-cell interactions. First, we established that droplet-based culture of microglia in the presence of LPS recapitulates the transcriptional effects of microglial activation with LPS in culture plates (data not shown). In addition, we confirmed that lentiviral transduction or puromycin treatment did not significantly alter microglial transcriptional responses associated with phagocytosis or other signaling pathways (data not shown).

Next, we confirmed that astrocyte treatment with a subthreshold dose of IL-1B/TNFα followed by incubation in droplets resulted in minimal background activation of NF-κB when astrocytes were co-incubated for 24 h either with control media or LPS-activated macrophages or microglia stably transduced with a non-targeting lentiviral CRISPR/Cas9 vector. FIGS. 6A-6G show Droplet screening control data. In FIG. 6, droplet cytometry plots show the gating strategy for unstimulated or non-targeting controls (FIGS. 6A-6B) and experimental (FIG. 6C) macrophage conditions as well as unstimulated or non-targeting controls (FIGS. 6D-6E) and experimental (FIG. 6F) microglia conditions at 24-hours post-encapsulation. FIG. 6G shows the quantification of the relative activation of reporter cells in droplets containing cell pairs that consist of macrophages or microglia together with astrocytes.

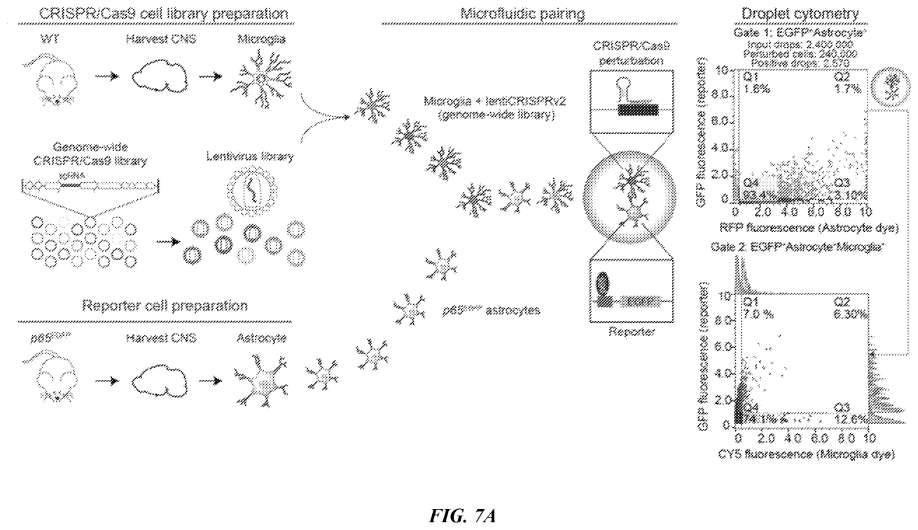

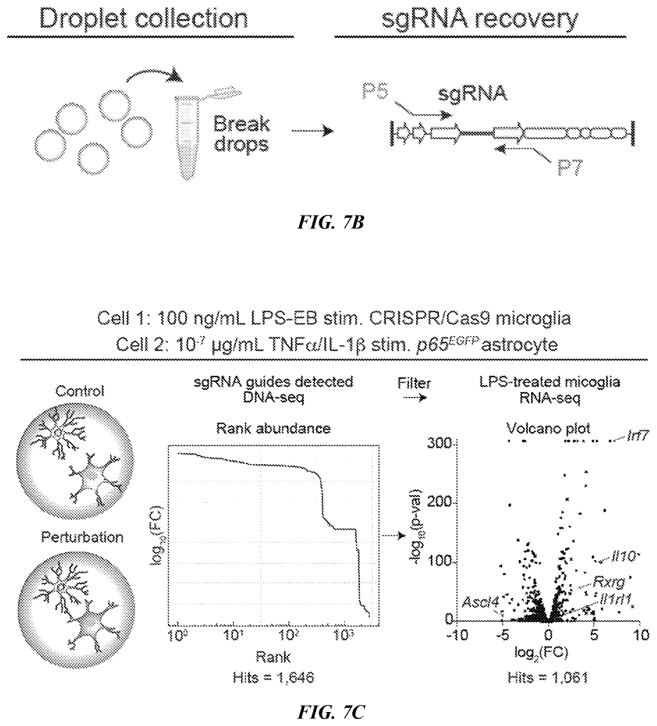

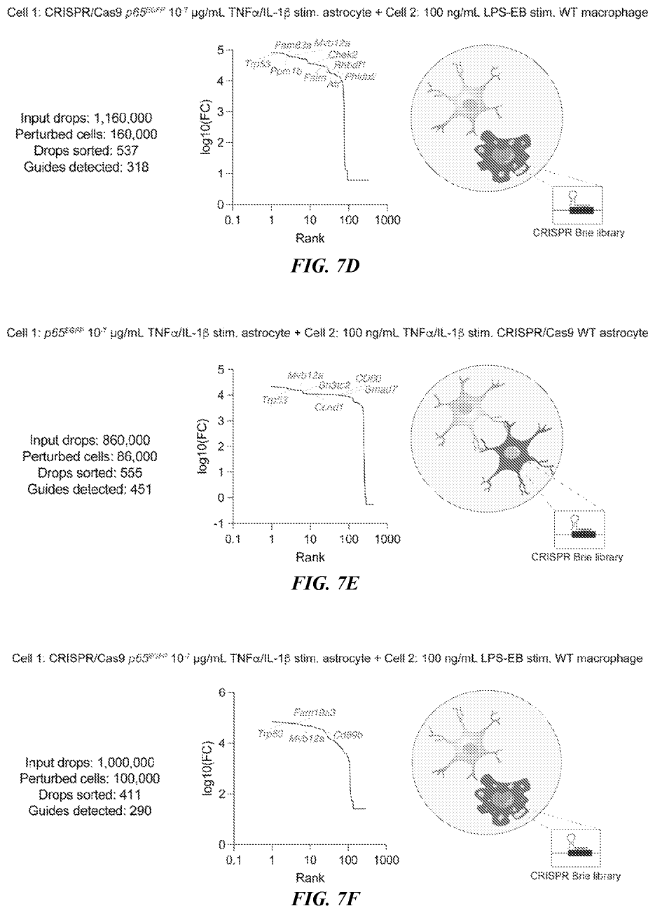

We used SPEAC-seq to identify microglial factors involved in the suppression of NF-κB activation in astrocytes because NF-κB is an important driver of disease-promoting astrocyte responses (2, 3, 5, 28). As shown in FIG. 7A, microglia were isolated from WT B6 mice and were stably transduced with puromycin with a pooled genome-wide lentiviral CRISPR/Cas9 library (78,637 sgRNA sequences) by low MOI spinfection to generate a single mutation in each cell. Astrocytes were isolated from p65^{EGFP} reporter mice and paired in droplets with a single CRISPR/Cas9 perturbed microglial cell for 24-hours. CRISPR/Cas9-based perturbations in microglia that resulted in NF-κB activation in astrocytes after 24-hours were screened using a high throughput microfluidic FACS platform. Identification of activated cell pairs after 24-hours using a 3-color, dual gating strategy. We calibrated droplet sorting gates to capture EGFP reporter activation in astrocytes paired with CRISPR/Cas9 transduced microglia compared to non-targeting controls. We also developed a workflow to isolate sgRNA sequences stably incorporated into microglial genomic DNA from small numbers of sorted droplets by genomic DNA PCR amplification and deep sequencing (FIG. 7B, showing that droplets are collected, broken and then the sgRNA were PCR amplified and sequenced), enabling the analysis of sgRNAs targeting microglial negative regulators of NF-κB activation in astrocytes. We filtered these SPEAC-seq hits against genes expressed in LPS-activated mouse microglia as detected by RNA-seq, resulting in a list of 1,061 candidate molecules (FIG. 7C, graphs showing analysis of guides detected (left) in the genomic DNA of microglia from sorted droplets containing an EGFP+ astrocyte (middle). SPEAC-seq hits were filtered against an RNA-seq database of LPS-activated primary mouse microglia (right). An analysis of the positive droplet fraction revealed known negative regulators of NF-κB signaling including xenobiotic metabolism (11, 30), nuclear receptor activation (31, 32), and NRF2 signaling (27), as well as high concordance between sorted drops and the number of guide RNA sequences detected. FIGS. 7D-7F are droplet screens. The left-side of each graph show the identification of genes based on positive EGFP cells. The middle is a schematic of the stimulation and pairing scheme; and the right is a gene ontology analysis of hits identified in each screen. P65^{EGFP} reporter astrocytes were incubated under the following conditions. Partner cells were transduced with a genome-wide CRISPR/Cas9 lentiviral library: (FIG. 7D) stimulated bone marrow-derived macrophages (24-hr of 100 ng/mL LPS-EB); (FIG. 7E) stimulated (24 hr of 100 ng/mL TNFα/IL-1β) astrocytes; (FIG. 7F) stimulated bone marrow-derived macrophages (24-hr of 100 ng/mL LPS-EB). See, also FIGS. 6G-6K. Conversely, the negative droplet fraction contained multiple non-targeting sgRNAs, highlighting the specificity of the droplet sorting procedure (see, FIG. 6K, which shows the fold enrichment of non-targeting sgRNAs detected in each of the EGFP+ or EGFP-fractions of p65^{EGFP} reporter astrocytes co-encapsulated with a perturbed microglial cell. n=17-26 sgRNAs per group).

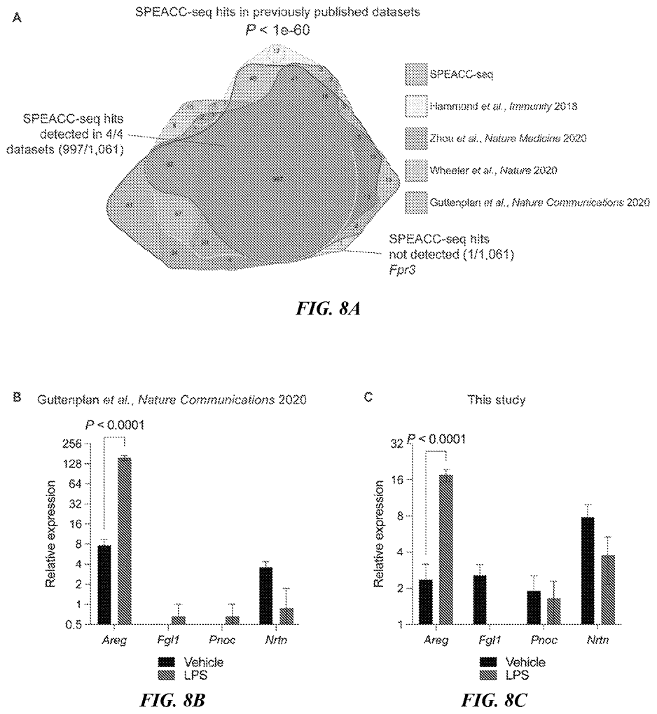

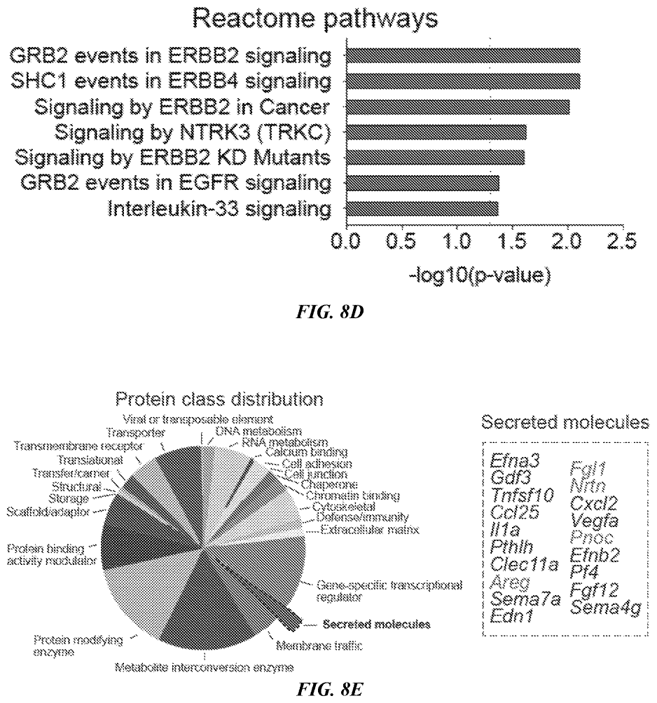

We then analyzed the SPEAC-seq dataset to identify microglial factors that suppress pro-inflammatory astrocyte responses. SPEAC-seq detected physiologically relevant candidate molecules expressed by microglia in four independent published bulk or scRNA-seq microglial datasets (27, 33-35). In FIG. 8A, the Venn diagram depicts the overlap of filtered SPEAC-seq hits filtered based on RNA expression from FIG. 7C with 4 independent bulk or single-cell RNA-seq datasets of microglia that were previously published (27, 34, 35, 78). All but one (1,060/1,061, 99.9%) of SPEAC-seq hits had been previously detected in microglia. A comparison of previously published data and the present study showed the sensitivity of the presently described methods (FIGS. 8B-8C. Next, we performed a gene ontology analysis of genes which, upon their CRISPR/Cas9-driven perturbation in microglia, led to NF-κB activation in astrocytes; these analyses identified transcriptional signatures linked to growth factor signaling. FIG. 8D shows the pathways detected by SPEAC-seq that limit astrocyte NF-κB activation discovered through bioinformatic analysis. To focus on candidate proteins involved in regulatory pathways that control cell-cell communication, we analyzed secreted molecules perturbed in microglia enriched in SPEAC-seq data and identified four candidate growth factors expressed by microglia (Areg, Nrtn, Fgl1, and Pnoc) (see, FIG. 8E) that signal through four independent receptors (Egfr, Lag3, Gfra2, Oprl1) expressed by astrocytes.

### Example 3: Microglia-Astrocyte AREG-EGFR Signaling Limits Astrocyte Pathogenic Activities in EAE

### Materials and Methods

### A. CRISPR/Cas9 Lentivirus Production

Lentiviral constructs were generated as previously described (12, 27, 37). The backbones used contain derivatives of the previously described reagents lentiCRISPR v2 (a gift from Feng Zhang, Addgene plasmid #52961 (79)), and lentiCas9-EGFP (a gift from Phil Sharp and Feng Zhang, Addgene plasmid #63592 (17)). Itgam-driven lentiviruses have been previously described (11, 12). Substitution of sgRNAs was performed through a PCR-based cloning strategy using Phusion Flash HF 2× Master Mix (Thermo Fisher, #F548L). A three-way cloning strategy was developed to substitute sgRNAs using the following primers: U6-PCR-F 5′-AAAGGCGCGCCGAGGGCCTATTT-3′ (SEQ ID NO:11), U6-PCR-R 5′-TTTTTTGGTCTCCCGGTGTTTCGTCCTTTCCAC-3′ (SEQ ID NO:12), cr-RNA-R 5′-GTTCCCTGCAGGAAAAAAGCACCGA-3′ (SEQ ID NO:13), cr-RNA-F 5′-AAAAAAGGTCTCTACCG (N₂₀) GTTTTAGAGCTAGAAATAGCAAGTT-3′ (SEQ ID NO: 14), where N₂₀ marks the sgRNA substitution site. The following sgRNA were designed using a combination of the Broad GPP sgRNA Designer Webtool (SpyoCas9, http://portals.broadinstitute.org/gpp/public/analysis-tools/sgrna-design), Synthego (https://design.synthego.com/#/), and cross-referenced with activity-optimized sequences contained within the Addgene library #1000000096 (a gift from David Sabatini and Eric Lander) (80). The sgRNA sequences used are as follows, with the promoter indicated in parentheses: sgScrmbl-5′-GCACTACCAGAGCTAACTCA-3′ (SEQ ID NO:15) (Itgam, Gfap); sgAreg 5′-AATGACCCCAGCTCAGGGAA-3′ (SEQ ID NO:16) (Itgam); sgligll-5′-CCAGTTTCTGGATAAAGGAT-3′ (SEQ ID NO:17) (Itgam); sgPnoc-5′-AGACCTTCTCTTCACACTGG-3′ (SEQ ID NO:18) (Itgam); sgNrtn-5′-GCTGGGCCTGGGCTACACGT-3′ (SEQ ID NO:19) (Itgam); sgEgfr-5′-GATGTACAACAACTGTGAAG-3′ (SEQ ID NO:20) (Gfap); sgGfra2-5′-GGCCAATAAGGAGTGCCAGG-3′ (SEQ ID NO:21) (Gfap); sgLag3-5′-GCCTGGGAAAGAGCTCCCCG-3′ (SEQ ID NO:22) (Gfap); and sgOprl1 5′-TGAGGATGACATACATGACG-3′ (SEQ ID NO:23) (Gfap). Amplicons were purified using the QIAquick PCR Purification Kit (Qiagen, #28104) and digested using DpnI (NEB, #R0176S), BsaI-HF (NEB, #R3535/R3733), AscI (for U6 fragment) (NEB, #R0558), or SbfI-HF (for crRNA fragment) (NEB, #R3642). pLenti backbone was cut with AscI/SbfI-HF and purified using the QIAquick PCR purification kit. Ligations into the respective backbone were performed overnight at 16° C. using T4 DNA Ligase Kit (NEB, #M0202L). Ligations were transformed into NEB Stable E. Coli (NEB, #C3040) at 42° C. and the ligation products were spread onto ampicillin selection plates. After overnight incubation at 37° C., single colonies were picked and DNA was prepared using QIAprep spin miniprep kit (Qiagen, #27104).

To generate barcoded lentiviral vectors, DNA oligonucleotides containing a barcode sequence were annealed into a degenerate dsDNA fragment with overhangs corresponding to 5′ BsrGI and 3′ EcoRI cut sites, which inserted barcodes immediately 3′ of the EGFP translational stop contained in Gfap::Cas9-2A-EGFP. Annealing was performed according to a protocol from Addgene by mixing 2 μg of each primer in 50 μL of annealing buffer (10 mM Tris pH=7.5, 50 mM NaCl, 1 mM EDTA), heating the tube to 95 C in a heat block, then moving the heat block to the bench top until it reached room temperature. The oligonucleotides used for this protocol were: FWD: 5′-GTACAAGTAANNNNNNNNGATGTCCACGAGGTCTCTGCTAGCG-3′ (SEQ ID NO: 24) and REV: 5′-AATTCGCTAGCAGAGACCTCGTGGACATCNNNNNNNNTTACTT-3′ (SEQ ID NO: 25) where NNNNNNNN represents the barcode sequence. Barcode sequences (5′->3′) used for this study were: sgScrmbl: CGTACTAG (SEQ ID NO:26), sgEgfr: CTCTCTAC (SEQ ID NO:27), sgGfra2: CAGAGAGG (SEQ ID NO:28), sgLag3: GCTACGCT (SEQ ID NO:29), and sgOprl1: CGAGGCTG (SEQ ID NO:30).

Lentiviral plasmids were transfected into HEK293FT cells according to the ViraPower Lentiviral Packaging Mix protocol (Thermo Fisher Scientific, #K497500) and lentiviruses were packaged with pLP1, pLP2, and pseudotyped with pLP/VSVG. Supernatant was aspirated the following day and fresh medium was added. After 2 days of incubation, lentivirus was collected and concentrated using Lenti-X Concentrator (Clontech, #631231) overnight at 4° C. followed by centrifugation according to the manufacturer's protocol. Lentiviral pellets were resuspended in 1/500 of the original volume and stored at −80° C.

### B. Intracranial Lentivirus Injection

C57Bl/6J mice at age 8-12 weeks were anesthetized using 1-3% isoflurane mixed with oxygen. Heads were shaved and cleaned using 70% ethanol and Betadine (Thermo Fisher, #19-027132) followed by a medial incision of the skin to expose the skull. The lateral ventricles were targeted bilaterally using the coordinates: +/−1.0 (lateral), −0.44 (posterior), −2.2 (ventral) relative to Bregma. Mice were injected with approximately 107 total IU of lentivirus delivered by two 10 μL injections using a 25 μL Hamilton syringe (Sigma-Aldrich, #20787) on a stereotaxic alignment system (Kopf, #1900) and the incision was sutured. Mice received 1 mg/kg Buprenorphine-SR via subcutaneous injection and were permitted to recover 7 days in a separate clean cage before induction of EAE. For Perturb-seq experiments, an equimolar cocktail of each barcoded lentivirus was injected +1.25 (lateral), +1.0 (rostral), −3.0 (ventral) relative to Bregma. For in vivo AREG treatment, mice were injected with 100 ng of AREG (R&D Systems, #989-AR-100) in 5 μL PBS or 5 μL of vehicle using the coordinates: +/−1.0 (lateral), −0.44 (posterior), −2.2 (ventral) relative to Bregma.

### C. EAE Induction:

EAE was induced with 150 μg of MOG₃₅₋₅₅ (Genemed Synthesis Inc., #110582) emulsified in freshly prepared complete Freund's adjuvant (Incomplete Freund's Adjuvant (BD Biosciences, #BD263910) mixed with Mycobacterium tuberculosis H-37Ra (BD Biosciences, #231141); final concentration 5 mg/ml). All animals received 2 subcutaneous injections of 100 μL each of MOG and a single intraperitoneal injection of 400 ng pertussis toxin (List Biological Laboratories, #180) in 200 μL of PBS. Mice received a second injection of pertussis toxin 48 hours after the initial injection. Mice were monitored and clinical scores were documented daily until the end of the experiment. Mice were sacrificed at different time points of disease. EAE clinical scores were defined as follows: 0-no signs, 1-fully limp tail, 2-hindlimb weakness, 3-hindlimb paralysis, 4-forelimb paralysis, 5-moribund.

### Results and Discussion

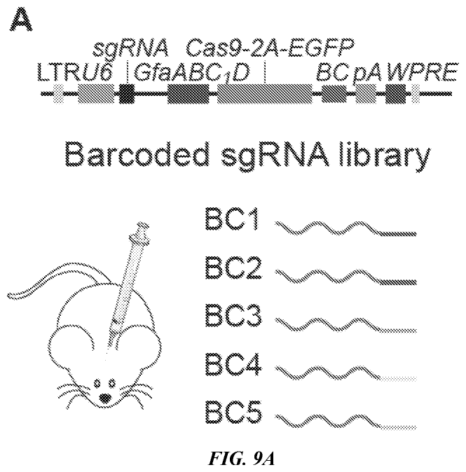

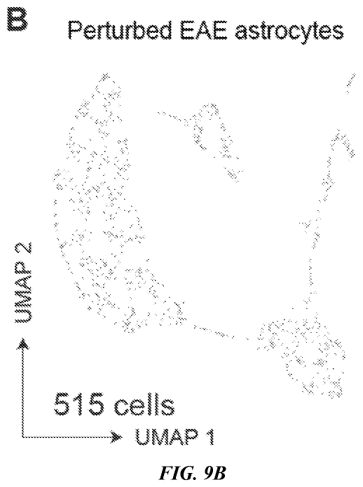

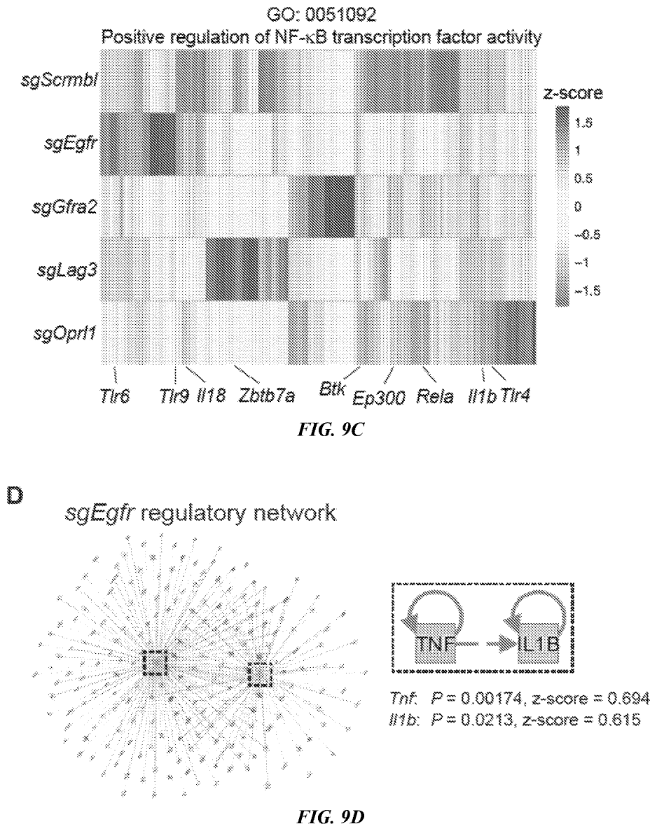

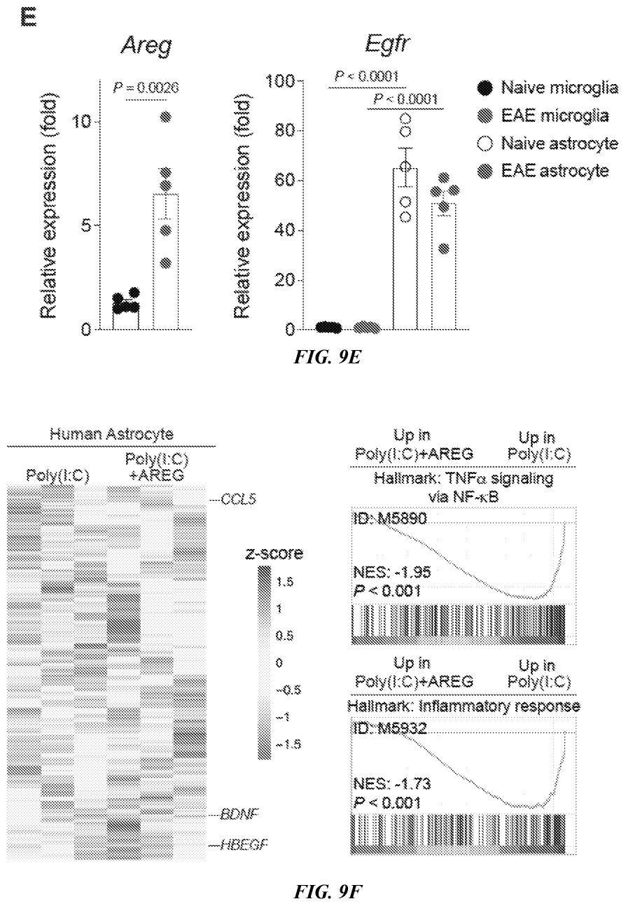

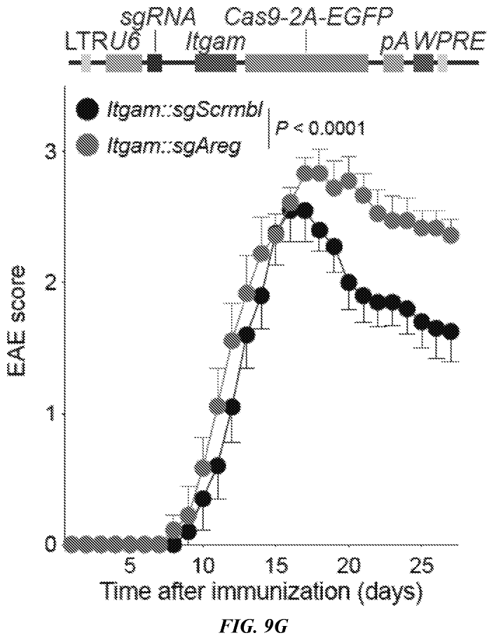

To evaluate the regulatory role of each candidate pathway uncovered by SPEAC-seq in the context of inflammation, we applied a cell type-specific in vivo Perturb-seq approach (36). We designed lentiviral vectors to co-express sgRNAs targeting receptors of interest with RNA-encoded barcodes in the Cas9 open reading frame (schematic shown in FIG. 9A); the expression of Cas9 and the RNA barcode was driven by the GFAP promoter active in astrocytes and detectable by scRNA-seq (12, 13, 27, 37). We designed an sgRNA against each receptor predicted to be activated by the microglial ligands identified by SPEAC-seq (Egfr, Gfra2, Lag3, Oprl1) as well as a non-targeting control, and used an equimolar cocktail of each virus to transduce the CNS of mice. After induction of EAE by immunization with MOG₃₅₋₅₅, we sorted EGFP+ astrocytes by flow cytometry, performed scRNA-seq and analyzed both the sgRNA and transcriptional profile of perturbed cells (FIG. 9B, showing a UMAP plot of astrocytes captured by Perturb-seq from EAE mice). When compared to the sgScrmbl control-transduced cells, astrocytes harboring sgRNAs targeting Egfr, Gfra2, Lag3, or Oprl1 showed increased NF-κB transcriptional activation (FIG. 9C, a heatmap showing the analysis of NF-κB signaling activation as a function of Perturb-seq-based knockdown of candidate astrocyte receptors). However, Egfr targeting led to the strongest activation of IL-1β/TNFα signaling, which promotes NF-κB-driven transcriptional astrocyte responses associated with EAE and MS (27) (FIG. 9D, a Qiagen IPA network analysis showing that EGFR signaling limits TNFα and IL-1β-driven NF-κB signals). Moreover, Egfr also showed higher expression in astrocytes in previously published scRNA-seq datasets than Gfra2, Lag3, and Oprl1 (27). The EGFR ligand identified by SPEAC-seq was Areg, which encodes amphiregulin. Notably, Areg showed higher expression than Nrtn, Fgl1 and Pnoc in stimulated mouse microglia. Thus, we investigated the effects of AREG-EGFR signaling on microglia-astrocyte interactions.

Amphiregulin is reported to control inflammation in the periphery (38-42) and in the CNS during stroke (43), suggesting that it is induced in response to trauma and/or inflammation. Indeed, we detected increased Areg expression in microglia at peak EAE, 17 days after disease induction (FIG. 9E, Egfr and Areg expression determined by qPCR in primary astrocytes and microglia from naïve or EAE mice). Consistent with our SPEAC-seq data, Egfr was expressed at higher levels in astrocytes than in microglia (FIG. 9E). We validated these findings by immunostaining, detecting the upregulation of microglial AREG levels during EAE (data not shown). The microglial expression of the microbiome-controlled EGFR ligand TGFα was reduced during EAE (11) (data not shown), suggesting a role for Areg+ microglia in limiting immunopathology during CNS inflammation. Indeed, the comparison of Areg+ and Tgfa+ microglia by scRNA-seq and RABID-seq (12) revealed largely non-overlapping microglial populations with unique transcriptional signatures and upstream regulators, which participated in different cell-cell interaction networks (data not shown).

To evaluate the functional impact of AREG signaling in astrocytes, we first evaluated the effect of AREG-treatment on primary mouse or human astrocytes activated with pro-inflammatory cytokines in vitro. In both serum-free and serum-containing mouse or human astrocytes, AREG decreased the activation of pro-inflammatory pathways associated with EAE and MS pathogenesis detected by qPCR and bulk RNA-seq (FIG. 9F, showing analysis of the transcriptional effects of AREG in human astrocytes pre-treated with pro-inflammatory cytokines and recombinant AREG). We detected a similar anti-inflammatory effect of AREG in astrocytes grown in adherent cultures or in droplets, or when astrocytes were isolated following AREG intracranial injection (data not shown).

To investigate the function of Areg+ microglia in vivo, we used a CRISPR/Cas9-expressing lentivirus under the control of the Itgam promoter administered via intracerebroventricular injection one week before EAE induction (11, 12). Consistent with an anti-inflammatory role for microglial AREG, sgAreg-targeted mice displayed a significant worsening of EAE when compared to controls (FIG. 9G, shows EAE disease course in mice transduced with Itgam::Cas9 lentiviruses co-expressing sgAreg or sgScrmbl). No effects were detected in the number of CNS-resident cells, recruited pro-inflammatory monocytes, nor CNS-recruited or splenic T cell subsets (data not shown), despite increased activation of microglia following Areg inactivation (data not shown), suggesting that AREG+ microglia limit local pro-inflammatory signals within the CNS. Moreover, the genetic inactivation of Fgl1, Nrtn, or Pnoc in microglia did not alter EAE development (data not shown). Similarly, EAE was not modified in Cd4::Cre; Areg(f/f) mice, in line with previous reports of Areg knockdown in regulatory T cells (44), while Areg−/− complete knockout mice displayed a worsening of EAE similar to the one detected following microglia-specific Areg knockdown (data not shown). Consistent with our SPEAC-seq data, astrocytes and microglia isolated from Itgam::sgAreg mice or Areg−/− mice and analyzed by RNA-seq or qPCR displayed increased NF-κB signaling relative to controls, concomitant with the activation of pathways associated with astrocyte pathogenic activities in EAE. Thus, microglial AREG signaling via EGFR suppresses astrocyte pathogenic activities during EAE, and potentially, MS.

### REFERENCES

1. D. S. Reich, C. F. Lucchinetti, P. A. Calabresi, Multiple Sclerosis. N Engl J Med 378, 169-180 (2018).
2. R. T. Han, R. D. Kim, A. V. Molofsky, S. A. Liddelow, Astrocyte-immune cell interactions in physiology and pathology. Immunity 54, 211-224 (2021).
3. M. Linnerbauer, M. A. Wheeler, F. J. Quintana, Astrocyte Crosstalk in CNS Inflammation. Neuron, (2020).
4. M. Prinz, T. Masuda, M. A. Wheeler, F. J. Quintana, Microglia and Central Nervous System-Associated Macrophages—From Origin to Disease Modulation. Annu Rev Immunol 39, (2021).
5. L. M. Sanmarco, C. M. Polonio, M. A. Wheeler, F. J. Quintana, Functional immune cell-astrocyte interactions. J Exp Med 218, (2021).
6. L. Schirmer, D. P. Schafer, T. Bartels, D. H. Rowitch, P. A. Calabresi, Diversity and Function of Glial Cell Types in Multiple Sclerosis. Trends Immunol 42, 228-247 (2021).
7. B. S. Khakh, B. Deneen, The Emerging Nature of Astrocyte Diversity. Annu Rev Neurosci 42, 187-207 (2019).
8. X. Yu, B. S. Khakh, SnapShot: Astrocyte interactions. Cell 185, 220-220 e221 (2022).
9. S. A. Liddelow et al., Neurotoxic reactive astrocytes are induced by activated microglia. Nature 541, 481-487 (2017).
10. I. D. Vainchtein et al., Astrocyte-derived interleukin-33 promotes microglial synapse engulfment and neural circuit development. Science 359, 1269-1273 (2018).
11. V. Rothhammer et al., Microglial control of astrocytes in response to microbial metabolites. Nature 557, 724-728 (2018).
12. I. C. Clark et al., Barcoded viral tracing of single-cell interactions in central nervous system inflammation. Science 372, (2021).
13. M. A. Wheeler et al., Environmental Control of Astrocyte Pathogenic Activities in CNS Inflammation. Cell 176, 581-596 e518 (2019).
14. A. Giladi et al., Dissecting cellular crosstalk by sequencing physically interacting cells. Nat Biotechnol, (2020).
15. R. Vento-Tormo et al., Single-cell reconstruction of the early maternal-fetal interface in humans. Nature 563, 347-353 (2018).
16. G. Pasqual et al., Monitoring T cell-dendritic cell interactions in vivo by intercellular enzymatic labelling. Nature 553, 496-500 (2018).
17. S. Chen et al., Genome-wide CRISPR screen in a mouse model of tumor growth and metastasis. Cell 160, 1246-1260 (2015).
18. R. D. Chow et al., In vivo profiling of metastatic double knockouts through CRISPR-Cpf1 screens. Nat Methods 16, 405-408 (2019).
19. M. W. LaFleur et al., A CRISPR-Cas9 delivery system for in vivo screening of genes in the immune system. Nat Commun 10, 1668 (2019).
20. R. T. Manguso et al., In vivo CRISPR screening identifies Ptpn2 as a cancer immunotherapy target. Nature 547, 413-418 (2017).
21. D. Pan et al., A major chromatin regulator determines resistance of tumor cells to T cell-mediated killing. Science 359, 770-775 (2018).
22. K. W. Wucherpfennig, A. N. Cartwright, Genetic screens to study the immune system in cancer. Curr Opin Immunol 41, 55-61 (2016).
23. H. Yin, W. Xue, D. G. Anderson, CRISPR-Cas: a tool for cancer research and therapeutics. Nat Rev Clin Oncol 16, 281-295 (2019).
24. A. Dixit et al., Perturb-Seq: Dissecting Molecular Circuits with Scalable Single-Cell RNA Profiling of Pooled Genetic Screens. Cell 167, 1853-1866 e1817 (2016).
25. I. C. Clark, R. Thakur, A. R. Abate, Concentric electrodes improve microfluidic droplet sorting. Lab on a Chip 18, 710-713 (2018).
26. M. B. Everhart et al., Duration and Intensity of NF-B Activity Determine the Severity of Endotoxin-Induced Acute Lung Injury. The Journal of Immunology 176, 4995-5005 (2006).
27. M. A. Wheeler et al., MAFG-driven astrocytes promote CNS inflammation. Nature 578, 593-599 (2020).
28. M. V. Sofroniew, Astrocyte barriers to neurotoxic inflammation. Nat Rev Neurosci 16, 249-263 (2015).
29. J. G. Doench et al., Optimized sgRNA design to maximize activity and minimize off-target effects of CRISPR-Cas9. Nat Biotechnol 34, 184-191 (2016).
30. V. Rothhammer et al., Type I interferons and microbial metabolites of tryptophan modulate astrocyte activity and central nervous system inflammation via the aryl hydrocarbon receptor. Nat Med 22, 586-597 (2016).
31. C. K. Glass, K. Saijo, Nuclear receptor transrepression pathways that regulate inflammation in macrophages and T cells. Nature Reviews Immunology 10, 365-376 (2010).
32. I. C. Clark et al., Identification of regulatory mechanisms operating in pathogenic astrocyte subsets using high throughput nucleic acid cytometry. Nature (In Press).
33. T. Masuda et al., Spatial and temporal heterogeneity of mouse and human microglia at single-cell resolution. Nature, (2019).
34. Y. Zhou et al., Human and mouse single-nucleus transcriptomics reveal TREM2-dependent and TREM2-independent cellular responses in Alzheimer's disease. Nat Med 26, 131-142 (2020).
35. T. R. Hammond et al., Single-Cell RNA Sequencing of Microglia throughout the Mouse Lifespan and in the Injured Brain Reveals Complex Cell-State Changes. Immunity, (2018).
36. X. Jin et al., In vivo Perturb-Seq reveals neuronal and glial abnormalities associated with autism risk genes. Science 370, (2020).
37. L. M. Sanmarco et al., Gut-licensed IFNγ (+) NK cells drive LAMP1 (+) TRAIL (+) anti-inflammatory astrocytes. Nature 590, 473-479 (2021).
38. L. A. Monticelli et al., IL-33 promotes an innate immune pathway of intestinal tissue protection dependent on amphiregulin-EGFR interactions. Proc Natl Acad Sci USA 112, 10762-10767 (2015).
39. D. M. W. Zaiss, W. C. Gause, L. C. Osborne, D. Artis, Emerging functions of amphiregulin in orchestrating immunity, inflammation, and tissue repair. Immunity 42, 216-226 (2015).
40. S. Krishnan et al., Amphiregulin-producing gammadelta T cells are vital for safeguarding oral barrier immune homeostasis. Proc Natl Acad Sci USA 115, 10738-10743 (2018).
41. C. M. Minutti et al., A Macrophage-Pericyte Axis Directs Tissue Restoration via Amphiregulin-Induced Transforming Growth Factor Beta Activation. Immunity 50, 645-654 e646 (2019).
42. A. M. Tsou et al., Neuropeptide regulation of non-redundant ILC2 responses at barrier surfaces. Nature, (2022).
43. M. Ito et al., Brain regulatory T cells suppress astrogliosis and potentiate neurological recovery. Nature 565, 246-250 (2019).
44. J. Pohar et al., Antigen receptor-engineered Tregs inhibit CNS autoimmunity in cell therapy using nonredundant immune mechanisms in mice. Eur J Immunol 52, 1335-1349 (2022).
45. H. R. Jiang et al., IL-33 attenuates EAE by suppressing IL-17 and IFN-gamma production and inducing alternatively activated macrophages. Eur J Immunol 42, 1804-1814 (2012).
46. D. Yang, Z. Han, J. J. Oppenheim, Alarmins and immunity. Immunol Rev 280, 41-56 (2017).
47. S. P. Gadani, J. T. Walsh, I. Smirnov, J. Zheng, J. Kipnis, The glia-derived alarmin IL-33 orchestrates the immune response and promotes recovery following CNS injury. Neuron 85, 703-709 (2015).
48. D. He et al., Disruption of the IL-33-ST2-AKT signaling axis impairs neurodevelopment by inhibiting microglial metabolic adaptation and phagocytic function. Immunity 55, 159-173 e159 (2022).
49. M. Funakoshi-Tago et al., TRAF6 is a critical signal transducer in IL-33 signaling pathway. Cell Signal 20, 1679-1686 (2008).
50. B. Demaree et al., Joint profiling of DNA and proteins in single cells to dissect genotype-phenotype associations in leukemia. Nat Commun 12, 1583 (2021).
51. A. M. Klein et al., Droplet barcoding for single-cell transcriptomics applied to embryonic stem cells. Cell 161, 1187-1201 (2015).
52. E. Z. Macosko et al., Highly Parallel Genome-wide Expression Profiling of Individual Cells Using Nanoliter Droplets. Cell 161, 1202-1214 (2015).
53. M. Stoeckius et al., Simultaneous epitope and transcriptome measurement in single cells. Nat Methods 14, 865-868 (2017).
54. R. A. Goodnow, Jr., C. E. Dumelin, A. D. Keefe, DNA-encoded chemistry: enabling the deeper sampling of chemical space. Nat Rev Drug Discov 16, 131-147 (2017).
55. W. G. Cochrane et al., Activity-Based DNA-Encoded Library Screening. ACS Comb Sci 21, 425-435 (2019).
56. D. Y. Kwon, Y. T. Zhao, J. M. Lamonica, Z. Zhou, Locus-specific histone deacetylation using a synthetic CRISPR-Cas9-based HDAC. Nat Commun 8, 15315 (2017).
57. S. Konermann et al., Genome-scale transcriptional activation by an engineered CRISPR-Cas9 complex. Nature 517, 583-588 (2015).
58. N. C. Yeo et al., An enhanced CRISPR repressor for targeted mammalian gene regulation. Nat Methods 15, 611-616 (2018).
59. O. O. Abudayyeh et al., RNA targeting with CRISPR-Cas13. Nature 550, 280-284 (2017).
60. K. A. Guttenplan et al., Neurotoxic Reactive Astrocytes Drive Neuronal Death after Retinal Injury. Cell Rep 31, 107776 (2020).
61. C. Schiering et al., The alarmin IL-33 promotes regulatory T-cell function in the intestine. Nature 513, 564-568 (2014).
62. N. Arpaia et al., A Distinct Function of Regulatory T Cells in Tissue Protection. Cell 162, 1078-1089 (2015).
63. D. Burzyn et al., A special population of regulatory T cells potentiates muscle repair. Cell 155, 1282-1295 (2013).
64. H. G. Lee, M. A. Wheeler, F. J. Quintana, Function and therapeutic value of astrocytes in neurological diseases. Nat Rev Drug Discov 21, 339-358 (2022).
65. B. Stevens et al., The classical complement cascade mediates CNS synapse elimination. Cell 131, 1164-1178 (2007).
66. M. K. Pandey et al., Complement drives glucosylceramide accumulation and tissue inflammation in Gaucher disease. Nature 543, 108-112 (2017).
67. A. Sekar et al., Schizophrenia risk from complex variation of complement component 4. Nature 530, 177-183 (2016).
68. S. Sawada, J. D. Scarborough, N. Killeen, D. R. Littman, A lineage-specific transcriptional silencer regulates CD4 gene expression during T lymphocyte development. Cell 77, 917-929 (1994).
69. R. Srinivasan et al., New Transgenic Mouse Lines for Selectively Targeting Astrocytes and Studying Calcium Signals in Astrocyte Processes In Situ and In Vivo. Neuron 92, 1181-1195 (2016).
70. L. Madisen et al., A robust and high-throughput Cre reporting and characterization system for the whole mouse brain. Nat Neurosci 13, 133-140 (2010).
71. A. D. Garcia, N. B. Doan, T. Imura, T. G. Bush, M. V. Sofroniew, GFAP-expressing progenitors are the principal source of constitutive neurogenesis in adult mouse forebrain. Nat Neurosci 7, 1233-1241 (2004).
72. W. Y. Chen, J. Hong, J. Gannon, R. Kakkar, R. T. Lee, Myocardial pressure overload induces systemic inflammation through endothelial cell IL-33. Proc Natl Acad Sci USA 112, 7249-7254 (2015).
73. S. Yona et al., Fate mapping reveals origins and dynamics of monocytes and tissue macrophages under homeostasis. Immunity 38, 79-91 (2013).
74. C. Gutiérrez-Vázquez, F. J. Quintana, Protocol for in vitro analysis of pro-inflammatory and metabolic functions of cultured primary murine astrocytes. STAR Protoc 3, 101033 (2022).
75. J. Joung et al., Genome-scale CRISPR-Cas9 knockout and transcriptional activation screening. Nat Protoc 12, 828-863 (2017).
76. S. A. Stewart et al., Lentivirus-delivered stable gene silencing by RNAi in primary cells. RNA 9, 493-501 (2003).
77. W. Li et al., MAGeCK enables robust identification of essential genes from genome-scale CRISPR/Cas9 knockout screens. Genome Biol 15, 554 (2014).
78. K. A. Guttenplan et al., Knockout of reactive astrocyte activating factors slows disease progression in an ALS mouse model. Nat Commun 11, 3753 (2020).
79. N. E. Sanjana, O. Shalem, F. Zhang, Improved vectors and genome-wide libraries for CRISPR screening. Nat Methods 11, 783-784 (2014).
80. T. Wang et al., Gene Essentiality Profiling Reveals Gene Networks and Synthetic Lethal Interactions with Oncogenic Ras. Cell 168, 890-903 e815 (2017).
81. C. C. Chao et al., Metabolic Control of Astrocyte Pathogenic Activity via cPLA2-MAVS. Cell 179, 1483-1498 e1422 (2019).
82. L. C. Foo, Purification of rat and mouse astrocytes by immunopanning. Cold Spring Harb Protoc 2013, 421-432 (2013).
83. A. J. Thompson et al., Diagnosis of multiple sclerosis: 2017 revisions of the McDonald criteria. The Lancet Neurology 17, 162-173 (2018).
84. T. Dhaeze et al., CD70 defines a subset of proinflammatory and CNS-pathogenic TH1/TH17 lymphocytes and is overexpressed in multiple sclerosis. Cell Mol Immunol 16, 652-665 (2019).
85. B. Broux et al., Interleukin-26, preferentially produced by TH17 lymphocytes, regulates CNS barrier function. Neurology (R) neuroimmunology & neuroinflammation 7, (2020).
86. J. J. Trombetta et al., Preparation of Single-Cell RNA-Seq Libraries for Next Generation Sequencing. Curr Protoc Mol Biol 107, 4.22.21-17 (2014).
87. G. X. Zheng et al., Massively parallel digital transcriptional profiling of single cells. Nat Commun 8, 14049 (2017).
88. S. L. Wolock, R. Lopez, A. M. Klein, Scrublet: Computational Identification of Cell Doublets in Single-Cell Transcriptomic Data. Cell systems 8, 281-291 e289 (2019).
89. C. Hafemeister, R. Satija, Normalization and variance stabilization of single-cell RNA-seq data using regularized negative binomial regression. Genome Biol 20, 296 (2019).
90. A. Butler, P. Hoffman, P. Smibert, E. Papalexi, R. Satija, Integrating single-cell transcriptomic data across different conditions, technologies, and species. Nat Biotechnol 36, 411-420 (2018).
91. P. T. Nguyen et al., Microglial Remodeling of the Extracellular Matrix Promotes Synapse Plasticity. Cell 182, 388-403 e315 (2020).
92. G. Finak et al., MAST: a flexible statistical framework for assessing transcriptional changes and characterizing heterogeneity in single-cell RNA sequencing data. Genome Biol 16, 278 (2015).
93. A. Subramanian et al., Gene set enrichment analysis: a knowledge-based approach for interpreting genome-wide expression profiles. Proc Natl Acad Sci USA 102, 15545-15550 (2005).
94. J. G. Perez-Silva, M. Araujo-Voces, V. Quesada, nVenn: generalized, quasi-proportional Venn and Euler diagrams. Bioinformatics 34, 2322-2324 (2018).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for studying interactions between particles, the method comprising:
obtaining a plurality of first particles, wherein each member of the plurality of first particles comprises one or more members of a library of interest;
obtaining a plurality of second particles, each second particle comprising a reporter molecule;
producing a plurality of droplets by encapsulating both a single first particle and a single second particle within an individual droplet;
incubating the plurality of droplets to allow for at least one interaction to occur between the encapsulated first and second particles;
selecting droplets that contain a reporter signal corresponding to the at least one interaction; and
analyzing the selected droplets to study the at least one interaction.

2. The method of claim 1, wherein the library of interest is a genetic perturbation library, a phage display library, a library of chemically diverse small particles, a library of mutant versions of a protein, or a library of gene promoters or enhancers.

3. The method of claim 2, wherein the genetic perturbation library is a CRISPR/Cas9 library.

4. The method of any one of claims 1-3, wherein the first and second particles are cells and the second cell is a partner cell to the first cell.

5. The method of any one of claims 1-4, wherein the reporter molecule is located in the second particle.

6. The method of claim 5, wherein the reporter molecule enables optical detection.

7. The method of claim 6, wherein the reporter molecule is a fluorescent protein.

8. The method of claim 7, wherein the fluorescent protein is Green Fluorescent Protein (GFP).

9. The method of any one of claims 1-8, further comprising, prior to encapsulation, staining the first and second particles with a viability dye.

10. The method of any one of claims 1-9, further comprising staining the droplets with a reference dye.

11. The method of any one of claims 1-10, wherein the droplets are sorted using multi-color optics.

12. The method of claim 11, wherein the droplets are sorted using 3-color optics.

13. The method of claim 12, wherein the 3-color optics is used to select droplets based on size of a droplet, presence of an activated reporter within a droplet, and a presence of both of the two particle types within a single droplet.

14. The method of any one of claims 1-13, wherein the droplets are further sorted using dielectrophoresis, electromagnetism, fluid flow forces, or physical partitions.

15. The method of claim 14, wherein the droplets are sorted using electromagnetism.

16. The method of claim 15, wherein the droplets are sorted using dielectrophoresis.

17. The method of any one of claims 1-16, wherein analyzing the droplets comprises:
breaking the droplets;
isolating perturbations;
assessing a readout of the perturbations; and
filtering the perturbations.

18. The method of claim 17, wherein the droplets are broken through chemical means or physical means.

19. The method of claim 18, wherein the droplets are broken using a combination of at least one freeze-thaw cycle and perfluorooctanol (PFO).

20. The method of claim 18, wherein the droplets are broken by applying an electric field to break the droplets.

21. The method of claim 17, wherein assessing the readout comprises conducting PCR amplification and sequencing of the perturbations.

22. The method of claim 17, wherein filtering the perturbations comprises comparing the results from the readout against known datasets and identifying unique perturbations that have not yet been previously identified.
